(19) United States
(12) Patent Application Publication
(10) Pub. No.: US 2023/0169652 A1
(43) Pub. Date: Jun. 1, 2023

(54) Determining Chest Conditions from Radiograph Data via Machine Learning

(71) Applicant: Google LLC, Mountain View, CA (US)

(72) Inventors: Sahar Kazemzadeh, San Francisco, CA (US); Dong Jin Yu, Seoul (KR); Shahar Jamshy, Santa Clara, CA (US); Rory Pilgrim, San Mateo, CA (US); Zaid Isam Nabulsi, San Francisco, CA (US); Andrew Beckmann Sellergren, El Cerrito, CA (US); Yun Liu, Mountain View, CA (US); Shruthi Prabhakara, Sunnyvale, CA (US); Atilla Peter Kiraly, San Jose, CA (US)

(21) Appl. No.: 18/011,888

(22) PCT Filed: May 12, 2022
(86) PCT No.: PCT/US2022/028975

§ 371 Date: Dec. 21, 2022

Related U.S. Application Data
(60) Provisional application No. 63/188,791, filed on May 14, 2021.

(51) Int. Cl. G06T 7/00; G06N 3/0464; G06N 3/084; G16H 10/60; G16H 30/40; G16H 50/20; G16H 50/30; G06T 7/11; A61B 6/00

(52) U.S. Cl. CPC G06T 7/0012; G06N 3/0464; G06N 3/084; G16H 10/60; G16H 30/40; G16H 50/20; G16H 50/30; G06T 7/11; A61B 6/50; G06T 2207/10116; G06T 2207/30061; G06T 2207/20081; G06T 2207/20084

(57) ABSTRACT

Systems and methods for chest condition determination can leverage one or more machine-learned models to process radiograph data to determine risk data (e.g., a preliminary diagnosis). For example, systems and methods can utilize a pathology model to process a chest x-ray to generate a tuberculosis diagnosis. The one or more machine-learned models can segment the lungs, can detect features in the data, and can pool the segmentation and located features to determine the diagnosis.

### RELATED APPLICATIONS

This application claims priority to and the benefit of U.S. Provisional Patent Application No. 63/188,791, filed May 14, 2021. U.S. Provisional Patent Application No. 63/188,791 is hereby incorporated by reference in its entirety.

### FIELD

The present disclosure relates generally to processing radiograph data to determine chest conditions. More particularly, the present disclosure relates to processing a chest x-ray with one or more machine-learned models to predict the risk of active pulmonary tuberculosis.

### BACKGROUND

Tuberculosis and other chest conditions can cause major medical issues. However, access to proper testing can be limited by regional constraints, economical constraints, and/or workforce constraints. For example, impoverished countries can experience limited accessibility to proper testing due to the lack of medical professionals and the high cost of certain tests. Patients in impoverished countries are not the only patients experiencing a lack of accessibility. The cost of medical care and seeing a medical specialist can limit the ability of patients even in some of the richest countries in the world.

In particular, tuberculosis (TB) is one of the top 10 causes of death worldwide and disproportionately affects low-to-middle-income-countries. Though the World Health Organization (WHO) recommends chest radiographs (CXR) to facilitate TB screening efforts, and the means of acquiring CXRs are generally accessible, accessibility to professionals with specialized expertise in CXR interpretation poses a challenge to broad implementation of TB screening efforts in many parts of the world.

Moreover, tuberculosis can manifest and look different depending on various factors including other health conditions, demographics, and stage of disease. Even trained specialists can have difficulty identifying tuberculosis and other conditions in an x-ray. Moreover, for some conditions that can cause difficulty in detection such as HIV (human immunodeficiency virus), early detection can be all the more important due to the higher mortality rate for patients with HIV.

### SUMMARY

Aspects and advantages of embodiments of the present disclosure will be set forth in part in the following description, or can be learned from the description, or can be learned through practice of the embodiments.

One example aspect of the present disclosure is directed to a computer-implemented method. The method can include receiving, by a computing system including one or more processors, patient data. The patient data can include a chest x-ray of a patient. The method can include processing, by the computing system, the patient data with a machine-learned pathology model to generate a tuberculosis diagnosis for the patient. In some implementations, the machine-learned pathology model can be trained to segment chest x-ray data and generate the tuberculosis diagnosis based at least in part on a segmented portion of the chest x-ray data. The method can include providing, by the computing system, the tuberculosis diagnosis as an output.

In some implementations, processing, by the computing system, the patient data with the machine-learned pathology model to generate the tuberculosis diagnosis can include processing, by the computing system, the patient data with a segmentation model to generate pixel data descriptive of identified pixels found corresponding to a patient's lungs; processing, by the computing system, the patient data with a detection model to generate detection data descriptive of regions of the chest x-ray with detected features; and generating, by the computing system, a tuberculosis diagnosis with a classification model based at least in part on the pixel data and the detection data. The machine-learned pathology model can include an attention pooling sub-block. The machine-learned pathology model can be trained to have at least 90 percent sensitivity and at least 70 percent specificity. In some implementations, the machine-learned pathology model can include a deep learning system architecture trained on a plurality of training examples from a plurality of patients from a plurality of countries. The method can include determining, by the computing system, a follow-up action based at least in part on the tuberculosis diagnosis.

In some implementations, processing, by the computing system, the patient data with the machine-learned pathology model can include determining, by the computing system, attributions in the patient data and overlaying, by the computing system, the attributions on the chest x-ray of the patient to provide visual cues of suspicious areas. The tuberculosis diagnosis can be at least one of positive, negative, or non-tuberculosis abnormality.

Another example aspect of the present disclosure is directed to a computing system. The computing system can include one or more processors and one or more non-transitory computer readable media that collectively store instructions that, when executed by the one or more processors, cause the computing system to perform operations. The operations can include obtaining radiograph data. The radiograph data can include a radiograph of a patient. The operations can include processing the radiograph data with a segmentation model to generate segmentation data descriptive of a segment of the radiograph comprising one or more organs (i.e., not descriptive of portions of the radiograph other than the segment). The operations can include processing the radiograph data with a detection model to generate detection data descriptive of one or more located features in the radiograph. The operations can include processing the segmentation data and the detection data with a classification model to generate classification data. In some implementations, the classification data can be descriptive of a determined classification for the one or more located features. The operations can include determining a diagnosis (e.g., determining risk data descriptive of a predicted risk that the patient has a particular diagnosis) based at least in part on the classification data.

In some implementations, processing the segmentation data and the detection data with a classification model to generate classification data can include processing the segmentation data and the detection data with an attention pooling sub-block to generate pooled data and processing the pooled data with a diagnosis model to generate the diagnosis (e.g., determining risk data descriptive of a predicted risk that the patient has a particular diagnosis). The diagnosis model can include a convolutional neural network. In some implementations, the segmentation model can include a mask region based convolutional neural network. The detection model can include a residual neural network architecture. The classification model can include a convolutional neural network comprising a fully connected sub-block. In some implementations, at least one of the segmentation model or classification model can include an EfficientNet architecture. The segmentation data can include a feature map. In some implementations, the detection data can include one or more attention masks.

Another example aspect of the present disclosure is directed to one or more non-transitory computer readable media that collectively store instructions that, when executed by one or more processors, cause a computing system to perform operations. The operations can include obtaining a plurality of training examples. In some implementations, each training example can include radiograph training data and a respective training label. The radiograph training data and the respective training label can be descriptive of a patient with a tuberculosis diagnosis. The operations can include processing the radiograph training data with a machine-learned pathology model to generate risk data (e.g., risk data predicting a risk that the patient has a particular predicted diagnosis (e.g., a tuberculosis diagnosis)). In some implementations, the machine-learned pathology model can be trained to segment radiograph data and generate the risk data based at least in part on segmented radiograph data. The operations can include evaluating a loss function that evaluates a difference between the risk data and the respective training label and adjusting one or more parameters of the machine-learned pathology model based at least in part on the loss function.

In some implementations, the training examples can include a set of region-specific training examples. The training examples can include a set of HIV training examples and a set of non-HIV training examples, and the set of HIV training examples can include one or more tuberculosis positive examples and one or more tuberculosis negative examples. In some implementations, the machine-learned pathology model can include a segmentation model, a detection model, and a classification model, and adjusting one or more parameters of the pathology model based at least in part on the loss function can include adjusting one or more parameters of at least one of the segmentation model, the detection model, or the classification model.

Another example aspect of the present disclosure is directed to one or more non-transitory computer readable media that collectively store instructions that, when executed by one or more processors, cause a computing system to perform operations. The operations can include obtaining a plurality of training examples. In some implementations, each training example can include radiograph training data and a respective training label. The radiograph training data and the respective training label can be descriptive of a patient with a tuberculosis diagnosis. The operations can include processing the radiograph training data with a machine-learned pathology model to generate a predicted diagnosis. In some implementations, the machine-learned pathology model can be trained to segment radiograph data and generate a diagnosis based at least in part on segmented radiograph data. The operations can include evaluating a loss function that evaluates a difference between the predicted diagnosis and the respective training label and adjusting one or more parameters of the machine-learned pathology model based at least in part on the loss function.

Another example aspect of the present disclosure is directed to a computer-implemented method. The method can include receiving, by a computing system including one or more processors, patient data. The patient data can include a chest x-ray of a patient. The method can include processing, by the computing system, the patient data with a machine-learned pathology model to generate risk data predicting a risk that the patient has tuberculosis (e.g., as a preliminary tuberculosis diagnosis). In some implementations, the machine-learned pathology model can be trained to segment chest x-ray data and generate the risk data based at least in part on a segmented portion of the chest x-ray data. The method can include providing, by the computing system, the risk data as an output.

In some implementations, processing, by the computing system, the patient data with the machine-learned pathology model to generate the risk data can include processing, by the computing system, the patient data with a segmentation model to generate pixel data descriptive of identified pixels found corresponding to a patient's lungs; processing, by the computing system, the patient data with a detection model to generate detection data descriptive of regions of the chest x-ray with detected features; and generating, by the computing system, risk data with a classification model based at least in part on the pixel data and the detection data. The machine-learned pathology model can include an attention pooling sub-block. The machine-learned pathology model can be trained to have at least 90 percent sensitivity and at least 70 percent specificity. In some implementations, the machine-learned pathology model can include a deep learning system architecture trained on a plurality of training examples from a plurality of patients from a plurality of countries. The method can include determining, by the computing system, a follow-up action based at least in part on the risk data.

In some implementations, processing, by the computing system, the patient data with the machine-learned pathology model can include determining, by the computing system, attributions in the patient data and overlaying, by the computing system, the attributions on the chest x-ray of the patient to provide visual cues of suspicious areas. The risk data can be indicative of at least one of positive, negative, or non-tuberculosis abnormality.

Other aspects of the present disclosure are directed to various systems, apparatuses, non-transitory computer-readable media, user interfaces, and electronic devices.

These and other features, aspects, and advantages of various embodiments of the present disclosure will become better understood with reference to the following description and appended claims. The accompanying drawings, which are incorporated in and constitute a part of this specification, illustrate example embodiments of the present disclosure and, together with the description, serve to explain the related principles.

### BRIEF DESCRIPTION OF THE DRAWINGS

Detailed discussion of embodiments directed to one of ordinary skill in the art is set forth in the specification, which makes reference to the appended figures, in which:

FIG. 1A depicts a block diagram of an example computing system that performs radiograph processing according to example embodiments of the present disclosure.

FIG. 1B depicts a block diagram of an example computing device that performs radiograph processing according to example embodiments of the present disclosure.

FIG. 1C depicts a block diagram of an example computing device that performs radiograph processing according to example embodiments of the present disclosure.

FIG. 2 depicts a block diagram of an example pathology model according to example embodiments of the present disclosure.

FIG. 3 depicts a block diagram of an example computing device that performs radiograph processing according to example embodiments of the present disclosure.

FIG. 4 depicts a block diagram of an example pathology model according to example embodiments of the present disclosure.

FIG. 5 depicts an illustration of example pathology model results according to example embodiments of the present disclosure.

FIG. 6 depicts a flow chart diagram of an example method to perform chest condition diagnostics according to example embodiments of the present disclosure.

FIG. 7 depicts a flow chart diagram of an example method to perform chest condition diagnostics according to example embodiments of the present disclosure.

FIG. 8 depicts a flow chart diagram of an example method to perform pathology model training according to example embodiments of the present disclosure.

FIG. 9A depicts an illustration of example pathology model outputs according to example embodiments of the present disclosure.

FIG. 9B depicts an illustration of an example pathology model output according to example embodiments of the present disclosure.

Reference numerals that are repeated across plural figures are intended to identify the same features in various implementations.

### DETAILED DESCRIPTION

Overview

Generally, the present disclosure is directed to systems and methods for screening chest conditions (e.g., tuberculosis). Screening for chest conditions can be inaccessible for impoverished countries or countries with limited access to specialists. Receiving a chest x-ray can be relatively inexpensive, but the cost and time delay of human review can cause many issues for the patients potentially dealing with dangerous conditions such as tuberculosis. The systems and methods disclosed herein can leverage a deep learning system architecture comprising one or more machine-learned models to quickly and accurately screen chest x-ray data to determine a risk data (e.g., data descriptive of a diagnosis) predicting a risk that the patient has certain medical condition (e.g., tuberculosis). The risk data can be used for screening, as a preliminary diagnosis, or can be used as a final diagnosis. For this reason, the risk data can be referred to below simply as a diagnosis.

The systems and methods disclosed herein can obtain or receive radiograph data descriptive of a chest radiograph of a patient. One or more machine-learned models can process the radiograph data to generate a classification to determine the likelihood of whether a patient has a chest condition, such as tuberculosis. The one or more machine learned models can include a segmentation model to generate segmentation data descriptive of a segment of the radiograph including the lungs, a detection model to generate detection data descriptive of one or more located features in the radiograph, and a classification model to generate classification data based on the segmentation data and the detection data. The classification data can be descriptive of a determined classification for the one or more located features. That classification data can include a classification that can be used to determine a chest condition diagnosis. In some implementations, the systems and methods can be configured to specialize in tuberculosis diagnosis such that the chest condition diagnosis can be a tuberculosis diagnosis. The tuberculosis diagnosis may be a tuberculosis positive diagnosis, a tuberculosis negative diagnosis, or a tuberculosis negative diagnosis with a non-tuberculosis lung abnormality. In some implementations, the detection data and the segmentation data may be pooled with an attention pooling sub-block.

In some implementations, the one or more machine-learned models can be one or more pathology models and may include one or more models for determining a diagnosis. For example, in some implementations, the pathology model can include a segmentation model, a detection model, and a classification model. The segmentation model can be trained to process radiograph data to generate segmentation data descriptive of the pixels spanning the lungs. The detection model can be trained to process the radiograph data to generate detection data descriptive of located features in the radiograph. The classification model can then be trained to process the segmentation data and the detection data to generate classification data descriptive of a determined classification based on the located features in the lung region of the radiograph. In some implementations, the classification data can then be used to determine a tuberculosis diagnosis. The tuberculosis diagnosis can be a positive diagnosis indicating a high likelihood the patient has tuberculosis, a negative diagnosis indicating a low likelihood the patient has tuberculosis, or a non-tuberculosis abnormality diagnosis indicating an abnormality exists but there is a low likelihood the abnormality is tuberculosis.

In some implementations, a segmentation model can include a mask region based convolutional neural network. Moreover, the segmentation model may include a Mask RCNN with a ResNet backbone architecture. Based on the segmentation results, the chest x-rays can be cropped using a tight bounding box enclosing the lungs as the input for the classification model. The segmentation model can process the radiograph data of a patient (e.g., a chest x-ray) to generate segmentation data, which may include one or more feature maps.

The detection model can include a residual neural network (ResNet). Alternatively and/or additionally, the detection model can include a Single Shot MultiBox Detector (SSD) to create bounding boxes around potential TB-relevant imaging features. Based on the predicted bounding boxes, a probabilistic attention mask can be calculated as the final pooling layer in the classification model. In some implementations, the detection model can process the radiograph data of a patient (e.g., a chest x-ray) to generate detection data, which may include one or more attention masks.

The classification model can include an attention pooling sub-block to process the segmentation data and detection data to generate pooled data. The classification model can further include a diagnosis model to process the pooled data. The diagnosis model can process the pooled data to generate the tuberculosis diagnosis. In some implementations, the diagnosis model can include a convolutional neural network and/or can include one or more fully-connected neural network layers.

In some implementations, the classification model can include a combination of an EfficientNet architecture pre-trained on classifying chest x-rays as normal or abnormal with an attention pooling layer, or sub-block, and a fully-connected layer, or sub-block. The attention pooling sub-block can utilize the probabilistic attention mask generated from the detection model to perform a weighted average of the feature maps before feeding to the final fully connected layer. In some implementations, the classification model can classify chest x-rays into 1 of 3 classes: tuberculosis positive, tuberculosis negative but abnormal, and normal. The systems and methods disclosed herein can take the prediction score for the tuberculosis positive class as the output prediction for all tuberculosis-related analysis.

The EfficientNet architecture can include a baseline network by performing a neural architecture search using the AutoML MNAS framework, which can optimize both accuracy and efficiency (FLOPS). The resulting architecture can use mobile inverted bottleneck convolution (MBConv). The baseline network can then be scaled up to obtain a family of models. EfficientNet can leverage model scaling techniques to improve model accuracy and efficiency for scaling up the machine-learned models.

Training the individual components of the pathology model, or deep learning system, can be a multi-step process.

The segmentation model can be trained using lung segmentation masks from a United States dataset, another country dataset, or a mixed dataset from a plurality of countries.

Training the detection model can involve using radiologist-annotated bounding boxes around tuberculosis-indicative abnormalities (e.g., nodules, airspace opacities with cavitation, airspace opacities without cavitation, pleural effusion, granulomas, lymphadenopathy, and fibroproductive lung opacities). Both the detection and classification models can be trained using a different dataset from the segmentation model, the same training dataset, or a mixed dataset from a plurality of countries. The machine-learned models can be trained on a limited amount of labeled data by using the noisy-student semi-supervised learning approach to leverage a much larger set of unlabeled data. More specifically, the systems and methods can obtain “noisy” chest condition (e.g., tuberculosis) labels by running inference using the initial version of the machine-learned models on a dataset with a large amount of unlabeled chest x-rays. The data with generated labels can then be combined with the original dataset to train 6 classification models, which can then be ensembled by taking the mean of the scores. In some implementations, the noisy student technique can begin with training using labels that currently exist, then applying labels to a larger sample of examples, and can then self-creating labels for new examples.

When training the detection model, the systems and methods can use a dropout keep probability of 0.99, and augmentation included random cropping, rotation, flipping, jitter on the bounding boxes, multi-scale anchors, and a box matcher with intersection-over-union.

When training the classification model, the systems and methods can apply a dropout, with a dropout keep probability of 0.5. Moreover, the systems and methods can apply data augmentation such as horizontal flipping, random shears, and random deformations. The hyperparameters can be selected based on empirical performance on the tune sets.

In some implementations, training the one or more machine learned-models can involve obtaining a plurality of training examples. Each training example can include radiograph training data and a respective training label. The radiograph training data and the respective training label can be descriptive of a patient with a tuberculosis diagnosis. For example, the radiograph training data can be data descriptive of a chest x-ray of a patient with one or more features indicative of tuberculosis. The respective training label for that radiograph training data can include a tuberculosis positive training label. The plurality of training examples can include a mix of tuberculosis diagnosis including positive diagnosis, negative diagnosis with no abnormalities, and negative diagnosis with an abnormality. The systems and methods can include processing the radiograph training data with a pathology model to generate a predicted diagnosis. The predicted diagnosis can be descriptive of whether the patient is determined to have tuberculosis. The systems and methods can then evaluate a loss function that evaluates a difference between the predicted diagnosis and the respective training label. One or more parameters of the pathology model can be adjusted based at least in part on the loss function. In some implementations, the training examples can include region-specific training examples. For example, a set of training examples can include labeled datasets from a plurality of patients in India. The training examples may include region-specific training examples from a plurality of regions. In some implementations, the training examples can include one or more training examples from HIV positive patients. The systems and methods can include a plurality of training examples from a plurality of HIV positive patients and a plurality of training examples from a plurality of HIV negative patients.

The pathology model can include a segmentation model, a detection model, and a classification model, and adjusting the one or more parameters of the pathology model can include adjusting one or more parameters of at least one of the segmentation model, the detection model, or the classification model.

In order to get the proper balance of sensitivity and specificity, model selection can be important for replicating or enhancing the services provided by human specialists. For model selection (checkpoint selection and other hyperparameter optimization), the systems and methods can select models to maximize the area under a receiver operating characteristic curve (i.e., area under ROC curve, or AUC) corresponding to the range of radiologists' sensitivities in the tune sets. The approach can be used to help explicitly select models that were performant across the range of radiologist sensitivities, instead of potentially optimizing for ranges that were beyond the scope of customary clinical practice. In some implementations, the sensitivity and specificity for the machine-learned models can be adjusted based on the practice location the systems and methods are implemented in order to normalize or adjust for practice tendencies in that area (e.g., the United States radiologists can have a higher sensitivity rate but lower specificity rate, while Indian radiologists can have lower sensitivity rate but higher specificity rate).

The systems and methods may further include determining a follow-up action based at least in part on the diagnosis. For example, in some implementations, a follow-up test can be advised or recommended based on the tuberculosis diagnosis to confirm the results. The follow-up action may include contacting a human specialist for review of the data or for performing a follow-up examination. In some implementations, the patient may be sent home if a negative diagnosis with no abnormality is determined. The patient may be referred to a different test or specialist based on the diagnosis. For example, a positive diagnosis for any form of chest condition can lead to a referral to a medical specialist.

In some implementations, the machine-learned model can include a deep learning system, and the machine-learned model may be trained using chest radiographs from patients in a variety of countries. For example, in some implementations, the machine learned models can be trained using chest x-ray examples with labels from various countries spanning Africa, Asia, and Europe. To improve generalization, the systems and methods can incorporate large-scale chest x-ray pretraining, attention pooling, and semi-supervised learning via “noisy student.” The machine-learned models can then be evaluated on test sets from different countries all with confirmation via microbiology or nucleic acid amplification testing (NAAT). Two of the test sets (e.g., test sets from India and US) can be from sites independent of those used in training. The WHO targets 90 percent sensitivity and 70 percent specificity; therefore, the machine-learned models operating point can be prespecified to favor sensitivity over specificity. In some implementations, the systems and methods can involve training the machine-learned models until the machine-learned models output diagnosis with at least 90 percent sensitivity and at least 70 percent specificity.

Evaluating the one or more machine-learned models can involve the use of a variety of training examples from different regions of the world. Different regions of the world can experience higher rates of tuberculosis, which may appear in different ways based on the environment, demographics of the region, and medical factors. Therefore, evaluating the one or more machine-learned models based on region-specific occurrences can test if the machine-learned model can accurately determine positive tests and negative tests in different regions. In some implementations, one or more parameters can be adjusted based on the evaluation to ensure a more accurate and aware machine-learned model for a variety of patients. Moreover, in some implementations, the machine-learned models can be evaluated with a set of radiograph examples with HIV and a set of radiograph examples without HIV, in order to evaluate the machine-learned models' ability to diagnose patients that may have a condition that can alter how tuberculosis manifests.

The systems and methods can provide performance that tracks closely with or exceeds radiologist specialists from various countries, with the clearest evidence of this trend seen in the enriched datasets which exhibit a marked rightward shift toward lower specificity. This can suggest that the inherent advantageous ability of the deep learning system to provide continuous “scores” as output for thresholding can likely help individual sites customize the triggering rate to their local practice patterns and resource needs, while trusting that the customized operating point has a similar effect to calibrating the “conservativeness” of a radiologist. The ability to calibrate the operating point may be critical over time even for the same population, as prevalence and disease severity can change over time. Statistical methods to tune operating points for each dataset and to detect when operating points should be updated over time may be useful in this regard.

The machine-learned models can be trained or evaluated on different types of chest conditions including different types of a certain condition and may be trained and evaluated on different stages and different levels of severity of the conditions. Evaluating the machine-learned models can involve evaluating differences in positive results and negative results for different datasets, which can be used to adjust one or more parameters of the machine-learned models.

The systems and methods disclosed herein can be utilized for prescreening, tuberculosis diagnosis, chest condition diagnosis, or general diagnosis for any body part. The pathology model can process an x-ray or radiograph data to generate a diagnosis. The pathology model can utilize a segmentation model to detect and segment the area of interest (e.g., an organ, a bone, etc.). The pathology model can include a detection model to get bounding boxes of features in the x-ray or radiograph, and the pathology model can utilize an attention pooling block to pool the data collected from the segmentation model and the detection model. The attention pooling can be used to generate a prediction with a classification model or a diagnosis model. For example, an x-ray of a patient's abdomen can be processed to determine if the patient has intestinal abnormalities and may diagnose the patient as having intestinal cancer. In some implementations, a plurality of separate models (e.g., 6 models) can be trained and then used to generate predictions. The predictions can then be averaged to provide a final prediction.

In some implementations, the systems and methods disclosed herein can be applied to processing other forms of medical imagery from a variety of imaging instruments (e.g., computed tomography scan, magnetic resonance imaging, ultrasound, nuclear medicine imaging, etc.).

The systems and methods of the present disclosure provide a number of technical effects and benefits. As one example, the system and methods can provide a tuberculosis diagnosis. More specifically, the systems and methods can process radiograph data with one or more machine-learned models to generate a tuberculosis diagnosis. The tuberculosis diagnosis can be completed as a preliminary review or may provide diagnosis for patients who have a lack of accessibility to medical specialists.

Another technical benefit of the systems and methods of the present disclosure is the ability to leverage machine-learned models to flag abnormalities in a patient's lungs. For example, a patient can receive a radiograph for chest condition screening. The radiograph can be processed by one or more machine-learned models to generate a preliminary report on whether the patient has any abnormalities. If an abnormality is flagged by the preliminary report, the patient can be informed that they should seek out further testing. The preliminary screening can be especially important for impoverished countries. Molecular tests for tuberculosis and other conditions can be expensive and obtaining a large amount of tests may be impractical for impoverished countries. Chest x-rays in comparison can be relatively inexpensive. The systems and methods disclosed herein can capitalize on the more accessible data to provide guidance on whether the patient should seek out the molecular test. The systems and methods disclosed herein can also avoid the high cost of medical specialists.

Another example of technical effect and benefit relates to improved computational efficiency and improvements in the functioning of a computing system. For example, the systems and methods disclosed herein can leverage the training of a segmentation model, a detection model, and a classification model to provide accurate diagnosis with limited training data. The trained models can be trained on a small training dataset and can accurately diagnose patients from throughout the world with various tangential issues that can cause experimental issues even without specialized training examples.

With reference now to the Figures, example embodiments of the present disclosure will be discussed in further detail.

Example Devices and Systems

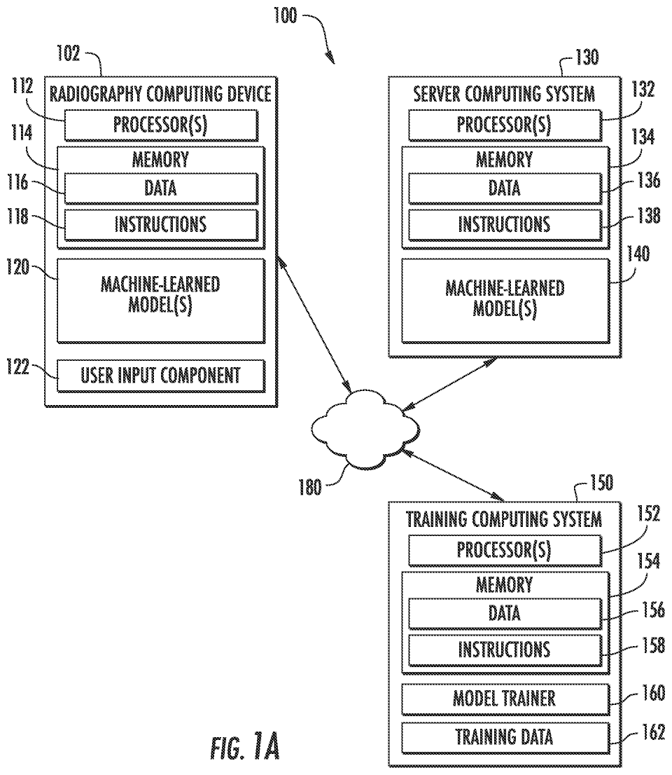

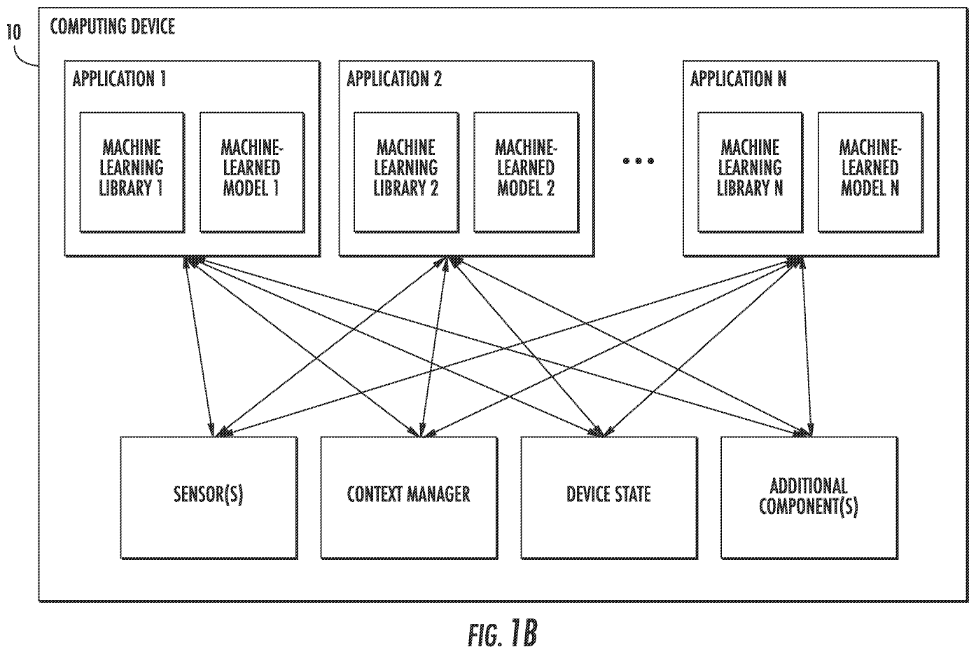

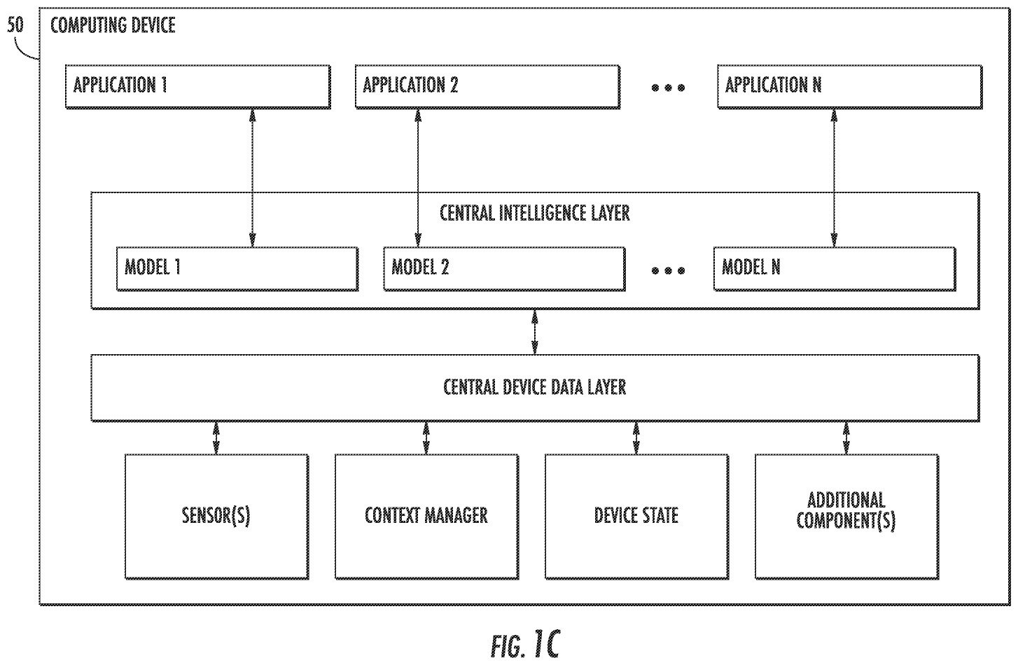

FIG. 1A depicts a block diagram of an example computing system 100 that performs radiograph data processing for chest condition diagnosis according to example embodiments of the present disclosure. The system 100 includes a user computing device 102, a server computing system 130, and a training computing system 150 that are communicatively coupled over a network 180.

The user computing device 102 can be any type of computing device, such as, for example, a personal computing device (e.g., laptop or desktop), a mobile computing device (e.g., smartphone or tablet), a gaming console or controller, a wearable computing device, an embedded computing device, or any other type of computing device.

The user computing device 102 includes one or more processors 112 and a memory 114. The one or more processors 112 can be any suitable processing device (e.g., a processor core, a microprocessor, an ASIC, a FPGA, a controller, a microcontroller, etc.) and can be one processor or a plurality of processors that are operatively connected. The memory 114 can include one or more non-transitory computer-readable storage mediums, such as RAM, ROM, EEPROM, EPROM, flash memory devices, magnetic disks, etc., and combinations thereof. The memory 114 can store data 116 and instructions 118 which are executed by the processor 112 to cause the user computing device 102 to perform operations.

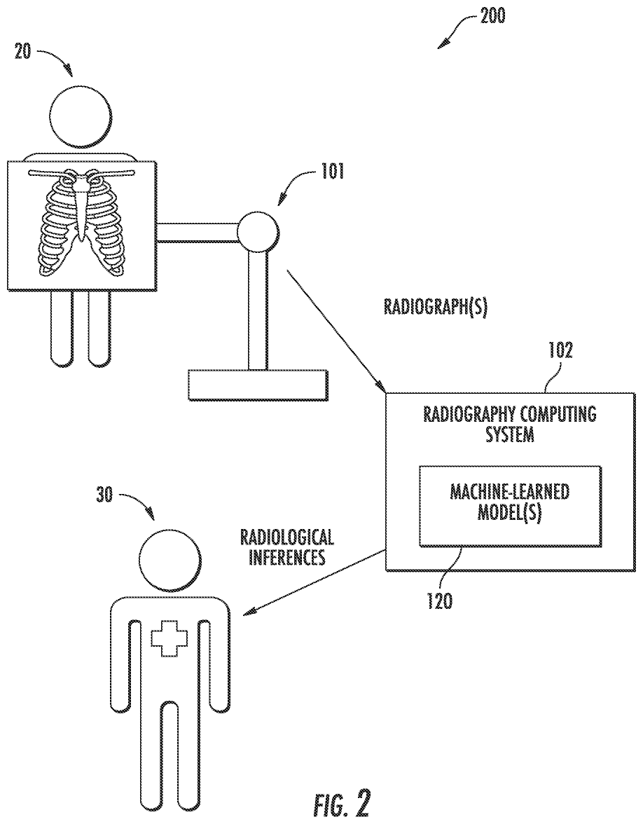

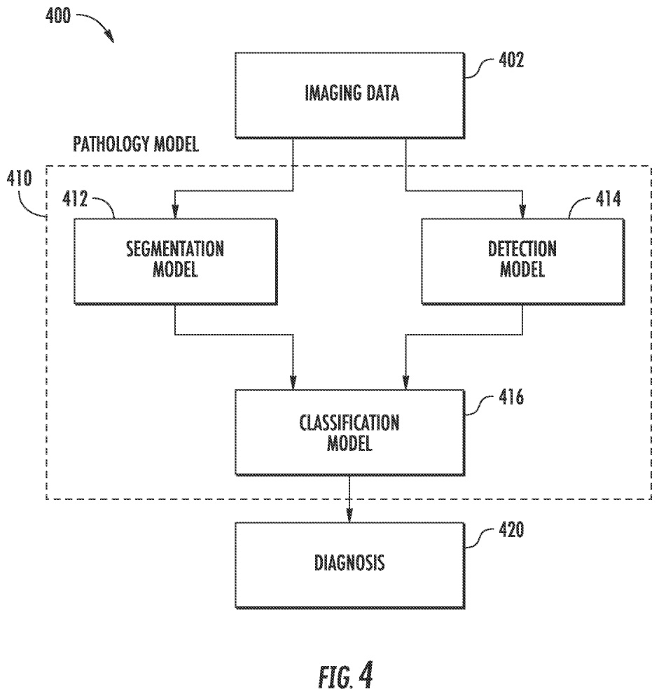

In some implementations, the user computing device 102 can store or include one or more pathology models 120. For example, the pathology models 120 can be or can otherwise include various machine-learned models such as neural networks (e.g., deep neural networks) or other types of machine-learned models, including non-linear models and/or linear models. Neural networks can include feed-forward neural networks, recurrent neural networks (e.g., long short-term memory recurrent neural networks), convolutional neural networks or other forms of neural networks. Example pathology models 120 are discussed with reference to FIGS. 2 and 4.

In some implementations, the one or more pathology models 120 can be received from the server computing system 130 over network 180, stored in the user computing device memory 114, and then used or otherwise implemented by the one or more processors 112. In some implementations, the user computing device 102 can implement multiple parallel instances of a single pathology model 120 (e.g., to perform parallel screening across multiple instances of radiograph data).

More particularly, the pathology model can process radiograph data to generate a diagnosis. Generation of the diagnosis can involve the use of a plurality of sub-models including a segmentation model, a detection model, and a classification model. The segmentation model can process the radiograph data to segment the lungs, the detection model can detect features in the radiograph data, and the classification model can pool the segmentation data and the detection model to use for determining a diagnosis.

Additionally or alternatively, one or more pathology models 140 can be included in or otherwise stored and implemented by the server computing system 130 that communicates with the user computing device 102 according to a client-server relationship. For example, the pathology models 140 can be implemented by the server computing system 140 as a portion of a web service (e.g., a screening service). Thus, one or more models 120 can be stored and implemented at the user computing device 102 and/or one or more models 140 can be stored and implemented at the server computing system 130.

The user computing device 102 can also include one or more user input component 122 that receives user input. For example, the user input component 122 can be a touch-sensitive component (e.g., a touch-sensitive display screen or a touch pad) that is sensitive to the touch of a user input object (e.g., a finger or a stylus). The touch-sensitive component can serve to implement a virtual keyboard. Other example user input components include a microphone, a traditional keyboard, or other means by which a user can provide user input.

The server computing system 130 includes one or more processors 132 and a memory 134. The one or more processors 132 can be any suitable processing device (e.g., a processor core, a microprocessor, an ASIC, a FPGA, a controller, a microcontroller, etc.) and can be one processor or a plurality of processors that are operatively connected. The memory 134 can include one or more non-transitory computer-readable storage mediums, such as RAM, ROM, EEPROM, EPROM, flash memory devices, magnetic disks, etc., and combinations thereof. The memory 134 can store data 136 and instructions 138 which are executed by the processor 132 to cause the server computing system 130 to perform operations.

In some implementations, the server computing system 130 includes or is otherwise implemented by one or more server computing devices. In instances in which the server computing system 130 includes plural server computing devices, such server computing devices can operate according to sequential computing architectures, parallel computing architectures, or some combination thereof.

As described above, the server computing system 130 can store or otherwise include one or more machine-learned pathology models 140. For example, the models 140 can be or can otherwise include various machine-learned models. Example machine-learned models include neural networks or other multi-layer non-linear models. Example neural networks include feed forward neural networks, deep neural networks, recurrent neural networks, and convolutional neural networks. Example models 140 are discussed with reference to FIGS. 2 and 4.

The user computing device 102 and/or the server computing system 130 can train the models 120 and/or 140 via interaction with the training computing system 150 that is communicatively coupled over the network 180. The training computing system 150 can be separate from the server computing system 130 or can be a portion of the server computing system 130.

The training computing system 150 includes one or more processors 152 and a memory 154. The one or more processors 152 can be any suitable processing device (e.g., a processor core, a microprocessor, an ASIC, a FPGA, a controller, a microcontroller, etc.) and can be one processor or a plurality of processors that are operatively connected. The memory 154 can include one or more non-transitory computer-readable storage mediums, such as RAM, ROM, EEPROM, EPROM, flash memory devices, magnetic disks, etc., and combinations thereof. The memory 154 can store data 156 and instructions 158 which are executed by the processor 152 to cause the training computing system 150 to perform operations. In some implementations, the training computing system 150 includes or is otherwise implemented by one or more server computing devices.

The training computing system 150 can include a model trainer 160 that trains the machine-learned models 120 and/or 140 stored at the user computing device 102 and/or the server computing system 130 using various training or learning techniques, such as, for example, backwards propagation of errors. For example, a loss function can be backpropagated through the model(s) to update one or more parameters of the model(s) (e.g., based on a gradient of the loss function). Various loss functions can be used such as mean squared error, likelihood loss, cross entropy loss, hinge loss, and/or various other loss functions. Gradient descent techniques can be used to iteratively update the parameters over a number of training iterations.

In some implementations, performing backwards propagation of errors can include performing truncated backpropagation through time. The model trainer 160 can perform a number of generalization techniques (e.g., weight decays, dropouts, etc.) to improve the generalization capability of the models being trained.

In particular, the model trainer 160 can train the pathology models 120 and/or 140 based on a set of training data 162. The training data 162 can include, for example, a plurality of training examples with training radiograph data and respective labels for the training radiograph data. In some implementations, the training examples can be indicative of patients from different areas of the world with different health statuses.

In some implementations, if the user has provided consent, the training examples can be provided by the user computing device 102. Thus, in such implementations, the model 120 provided to the user computing device 102 can be trained by the training computing system 150 on user-specific data received from the user computing device 102. In some instances, this process can be referred to as personalizing the model.

The model trainer 160 includes computer logic utilized to provide desired functionality. The model trainer 160 can be implemented in hardware, firmware, and/or software controlling a general purpose processor. For example, in some implementations, the model trainer 160 includes program files stored on a storage device, loaded into a memory and executed by one or more processors. In other implementations, the model trainer 160 includes one or more sets of computer-executable instructions that are stored in a tangible computer-readable storage medium such as RAM hard disk or optical or magnetic media.

The network 180 can be any type of communications network, such as a local area network (e.g., intranet), wide area network (e.g., Internet), or some combination thereof and can include any number of wired or wireless links. In general, communication over the network 180 can be carried via any type of wired and/or wireless connection, using a wide variety of communication protocols (e.g., TCP/IP, HTTP, SMTP, FTP), encodings or formats (e.g., HTML, XML), and/or protection schemes (e.g., VPN, secure HTTP, SSL).

The machine-learned models described in this specification may be used in a variety of tasks, applications, and/or use cases.

In some implementations, the input to the machine-learned model(s) of the present disclosure can be image data. The machine-learned model(s) can process the image data to generate an output. As an example, the machine-learned model(s) can process the image data to generate an image recognition output (e.g., a recognition of the image data, a latent embedding of the image data, an encoded representation of the image data, a hash of the image data, etc.). As another example, the machine-learned model(s) can process the image data to generate an image segmentation output. As another example, the machine-learned model(s) can process the image data to generate an image classification output. As another example, the machine-learned model(s) can process the image data to generate an image data modification output (e.g., an alteration of the image data, etc.). As another example, the machine-learned model(s) can process the image data to generate an encoded image data output (e.g., an encoded and/or compressed representation of the image data, etc.). As another example, the machine-learned model(s) can process the image data to generate an upscaled image data output. As another example, the machine-learned model(s) can process the image data to generate a prediction output.

In some implementations, the input to the machine-learned model(s) of the present disclosure can be latent encoding data (e.g., a latent space representation of an input, etc.). The machine-learned model(s) can process the latent encoding data to generate an output. As an example, the machine-learned model(s) can process the latent encoding data to generate a recognition output. As another example, the machine-learned model(s) can process the latent encoding data to generate a reconstruction output. As another example, the machine-learned model(s) can process the latent encoding data to generate a prediction output.

In some implementations, the input to the machine-learned model(s) of the present disclosure can be statistical data. The machine-learned model(s) can process the statistical data to generate an output. As an example, the machine-learned model(s) can process the statistical data to generate a recognition output. As another example, the machine-learned model(s) can process the statistical data to generate a prediction output. As another example, the machine-learned model(s) can process the statistical data to generate a classification output. As another example, the machine-learned model(s) can process the statistical data to generate a segmentation output. As another example, the machine-learned model(s) can process the statistical data to generate a segmentation output. As another example, the machine-learned model(s) can process the statistical data to generate a visualization output. As another example, the machine-learned model(s) can process the statistical data to generate a diagnostic output.

In some implementations, the input to the machine-learned model(s) of the present disclosure can be sensor data. The machine-learned model(s) can process the sensor data to generate an output. As an example, the machine-learned model(s) can process the sensor data to generate a recognition output. As another example, the machine-learned model(s) can process the sensor data to generate a prediction output. As another example, the machine-learned model(s) can process the sensor data to generate a classification output. As another example, the machine-learned model(s) can process the sensor data to generate a segmentation output. As another example, the machine-learned model(s) can process the sensor data to generate a segmentation output. As another example, the machine-learned model(s) can process the sensor data to generate a visualization output. As another example, the machine-learned model(s) can process the sensor data to generate a diagnostic output. As another example, the machine-learned model(s) can process the sensor data to generate a detection output.

In some cases, the input includes visual data and the task is a computer vision task. In some cases, the input includes pixel data for one or more images and the task is an image processing task. For example, the image processing task can be image classification, where the output is a set of scores, each score corresponding to a different object class and representing the likelihood that the one or more images depict an object belonging to the object class. The image processing task may be object detection, where the image processing output identifies one or more regions in the one or more images and, for each region, a likelihood that region depicts an object of interest. As another example, the image processing task can be image segmentation, where the image processing output defines, for each pixel in the one or more images, a respective likelihood for each category in a predetermined set of categories. For example, the set of categories can be foreground and background. As another example, the set of categories can be object classes. As another example, the image processing task can be depth estimation, where the image processing output defines, for each pixel in the one or more images, a respective depth value. As another example, the image processing task can be motion estimation, where the network input includes multiple images, and the image processing output defines, for each pixel of one of the input images, a motion of the scene depicted at the pixel between the images in the network input.

FIG. 1A illustrates one example computing system that can be used to implement the present disclosure. Other computing systems can be used as well. For example, in some implementations, the user computing device 102 can include the model trainer 160 and the training dataset 162. In such implementations, the models 120 can be both trained and used locally at the user computing device 102. In some of such implementations, the user computing device 102 can implement the model trainer 160 to personalize the models 120 based on user-specific data.

FIG. 1B depicts a block diagram of an example computing device 10 that performs according to example embodiments of the present disclosure. The computing device 10 can be a user computing device or a server computing device.

The computing device 10 includes a number of applications (e.g., applications 1 through N). Each application contains its own machine learning library and machine-learned model(s). For example, each application can include a machine-learned model. Example applications include a text messaging application, an email application, a dictation application, a virtual keyboard application, a browser application, etc.

As illustrated in FIG. 1B, each application can communicate with a number of other components of the computing device, such as, for example, one or more sensors, a context manager, a device state component, and/or additional components. In some implementations, each application can communicate with each device component using an API (e.g., a public API). In some implementations, the API used by each application is specific to that application.

FIG. 1C depicts a block diagram of an example computing device 50 that performs according to example embodiments of the present disclosure. The computing device 50 can be a user computing device or a server computing device.

The computing device 50 includes a number of applications (e.g., applications 1 through N). Each application is in communication with a central intelligence layer. Example applications include a text messaging application, an email application, a dictation application, a virtual keyboard application, a browser application, etc. In some implementations, each application can communicate with the central intelligence layer (and model(s) stored therein) using an API (e.g., a common API across all applications).

The central intelligence layer includes a number of machine-learned models. For example, as illustrated in FIG. 1C, a respective machine-learned model (e.g., a model) can be provided for each application and managed by the central intelligence layer. In other implementations, two or more applications can share a single machine-learned model. For example, in some implementations, the central intelligence layer can provide a single model (e.g., a single model) for all of the applications. In some implementations, the central intelligence layer is included within or otherwise implemented by an operating system of the computing device 50.

The central intelligence layer can communicate with a central device data layer. The central device data layer can be a centralized repository of data for the computing device 50. As illustrated in FIG. 1C, the central device data layer can communicate with a number of other components of the computing device, such as, for example, one or more sensors, a context manager, a device state component, and/or additional components. In some implementations, the central device data layer can communicate with each device component using an API (e.g., a private API).

FIG. 2 depicts an example computing system according to example embodiments of the present disclosure. In particular, FIG. 2 shows an alternative system where one or more machine-learned models 120 are employed by a radiography computing system 102 to generate the radiological inferences.

More particularly, in FIG. 2, an x-ray machine 101 can be operated to generate one or more radiographs that depict portions of a patient 20. The radiographs can be initially collected or provided to a radiography computing system 102. For example, the radiography computing system 102 can be a computing system that is on-site with the x-ray machine 101. For example, the radiography computing system 102 can be a portion of the x-ray machine 101 (e.g., the radiography computing system 102 can control the x-ray machine 101 and receive and store the x-ray data (e.g., radiographs) from the x-ray machine 101 upon capture). Alternatively or additionally, the radiography computing system 102 can be a separate system that is on-site at a medical care facility along with the x-ray machine 101. For example, the radiography computing system 102 can be a medical provider's computing system such as a computing system operated for a hospital, physician's office, and/or the like (e.g., which stores various types of patient files or data).

In some implementations, the radiographs are transmitted from the radiography computing system 102 to a remote radiograph interpretation system. For example, the remote radiograph interpretation system can be a cloud service (e.g., accessible via API(s)) to which the radiography computing system 102 can make calls to receive radiological inferences. Specifically, the remote radiograph interpretation system can store and use one or more machine-learned models 140 to generate one or more radiological inferences based on the radiograph(s). For example, each radiological inference can indicate the presence or absence (e.g., with some measure of confidence) of whether a given radiograph depicts a given condition. The remote radiograph interpretation system can transmit the radiological inference(s) to the radiography computing system 102 and the radiography computing system 102 can provide (e.g., display) the radiological inferences to a care provider 30 (e.g., physician or other medical professional). The care provider 30 can use the radiological inferences (e.g., in addition to their own judgment) to determine a diagnosis and/or treatment plan for the patient 20. In some implementations, the radiological inferences can specifically include a suggested treatment for the inferred conditions.

Moreover, FIG. 2 depicts that the radiographs are analyzed locally at the radiography computing system 102 using one or more machine-learned models 120 which are locally stored at the radiography computing system 102.

Example Model Arrangements

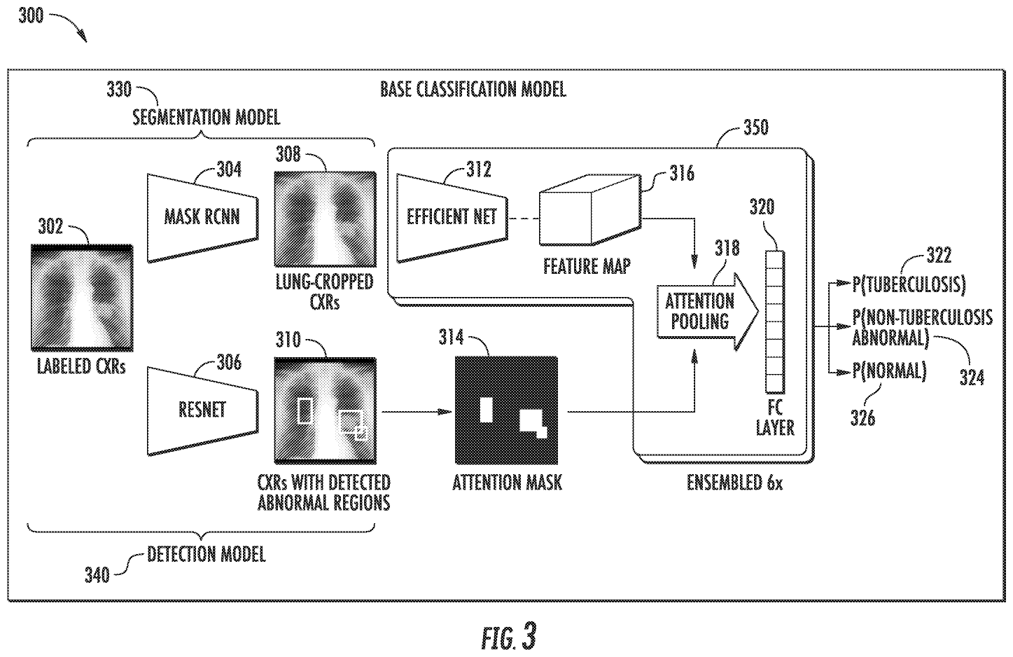

FIG. 3 depicts a block diagram of an example pathology model 300 according to example embodiments of the present disclosure. In some implementations, the pathology model 300 is trained to receive a set of input data 302 descriptive of a chest x-ray of a patient and, as a result of receipt of the input data 302, provide output data 322, 324, or 326 that is descriptive of a determined diagnosis. Thus, in some implementations, the pathology model 300 can include a classification model 350 that is operable to process the output of the segmentation model 330 and the detection model 340.

The pathology model 300 of FIG. 3 can include a segmentation model 330, a detection model 340, and a classification model 350. Moreover, the segmentation model 330 can include a Mask RCNN 304, the detection model 340 can include a ResNet architecture 306, and the classification model 350 can include attention pooling 318 and a fully convolutional sub-block 320.

In some implementations, the pathology model 300 can be trained on labeled chest x-rays 302. For example, the labeled x-rays 302 can be processed by the Mask RCNN 304 of the segmentation model 330 to generate lung-cropped chest x-rays 308. The labeled chest x-rays 302 can also be processed by the ResNet architecture 306 of the detection model 340 to generate chest x-rays with detected abnormal regions 310. The chest x-rays with detected abnormal regions 310 can be used to generate attention masks 314 based on the detected abnormal regions.

The lung-cropped chest x-rays 308 can then be processed with an EfficientNet sub-block 312 to generate a feature map 316 with normal and abnormal determinations. The feature map 316 and the attention masks 314 can then be pooled with an attention pooling block 318 and processed by a convolutional neural network with a fully convolutional sub-block 320. In some implementations, the classification model 350 can include an ensemble of a number of member models (e.g., six models in the ensemble). The output of the classification model can include a predicted tuberculosis diagnosis, which can include positive for tuberculosis 322, a non-tuberculosis abnormality 324, or normal 326. The predicted diagnosis can be evaluated against the label for the respective chest x-ray, and in response to the evaluation results, one or more parameters of the pathology model 300 may be adjusted.

In some implementations, the trained pathology model 300 can then process chest x-rays 302 or radiograph data to generate a diagnosis. For example, the radiograph data can be processed by the segmentation model 330 and the detection model 340 to generate segmentation data (e.g., lung-cropped chest x-rays 308 generated using the Mask RCNN 304) and detection data (e.g., bounding boxes 310 and/or attention masks 314 generated with the ResNet architecture 306), respectively. The segmentation data and detection data can then be processed by the classification model 350 to determine abnormalities, pool the data, and generate classification probabilities, which can be used for determining a diagnosis. For example, the classification model 350 can pool the segmentation data and the detection data with the attention pooling block 318, process the pooled data with a fully convolutional sub-block, and determine a likelihood the chest x-ray is indicative of tuberculosis 322, a non-tuberculosis abnormality 324, or normal. The classification with the highest likelihood or probability may be determined as the diagnosis. In some implementations, the systems and methods may require a certain level of certainty in order to provide a diagnosis. The level of certainty may be a threshold determined based at least in part on the region of practice, severity of condition, medical protocols, and/or preference. In some implementations, the threshold can be adjusted based on whether the pathology model 300 is being used for screening or actionable condition diagnosis.

FIG. 4 depicts a block diagram of an example pathology model 300 according to example embodiments of the present disclosure. The pathology model 300 is similar to the pathology model 300 of FIG. 3.

The pathology model 400 of FIG. 4 can be trained for diagnosis of a variety of conditions based on imaging data. In some implementations, the pathology model 400 can be trained for tuberculosis diagnosis, chest condition diagnosis, abdomen condition diagnosis, or general imaging diagnosis. The input can be imaging data 402 including, but not limited to, x-rays, ultrasounds, MRIs, etc. The imaging data 402 can be descriptive of the anatomy of a patient seeking screening or general diagnosis.

The imaging data 402 can be processed by a segmentation model 412 to locate and isolate an area of interest, such as an organ, ligament, or bone of interest. The segmentation model 412 can output segmentation data descriptive of the segmented imaging data (and not descriptive of parts of the imaging data outside the area of interest). The imaging data 402 can also be processed by a detection model 414 to detect features in the imaging data. The detection model 414 can output detection data descriptive of detected features, which can be denoted with bounding boxes and/or attention masks.

The segmentation data and the detection data can then be processed by a classification model 416 to pool the data and determine a classification. The classification can be a diagnosis 420 or may be used for determining the diagnosis 420. The diagnosis can be a binary positive or negative, and in some implementations, the diagnosis can be any number of possibilities.

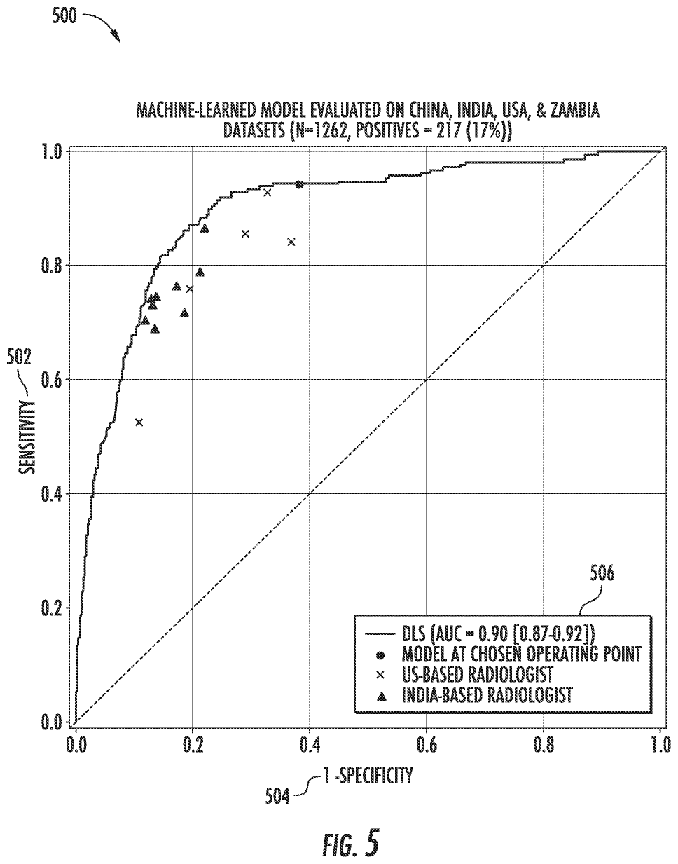

FIG. 5 depicts an illustration of example experimental results 500 according to example embodiments of the present disclosure. The example experimental results 500 can include a graph depicting the sensitivity 502 and the specificity 504 of an example pathology model, a plurality of India-based radiologists, and a plurality of US-based radiologists.

The graph in FIG. 5 depicts the performance of a set of India-based radiologists, a set of US-based radiologists, and an example deep learning system according to some implementations of the systems and methods. The set of India-based radiologists, the set of US-based radiologists, and the example deep learning system analyzed a set of data sets from four countries (i.e., China, India, U.S., and Zambia). The graph depicts the determined specificity 504 and sensitivity 502 based on their analysis and diagnosis. As referenced in the legend 506, the line represents the receiver operating characteristic (ROC) curve of the example deep learning system, the dot represents a model at a chosen operating point, the X's represent the various US-based radiologists in the set of US-based radiologists, and the triangles represent the various India-based radiologists in the set of India-based radiologists.

As conveyed in the graph, the example deep learning system performed similar to or better than the India-based radiologists and the US-based radiologists on the dataset.

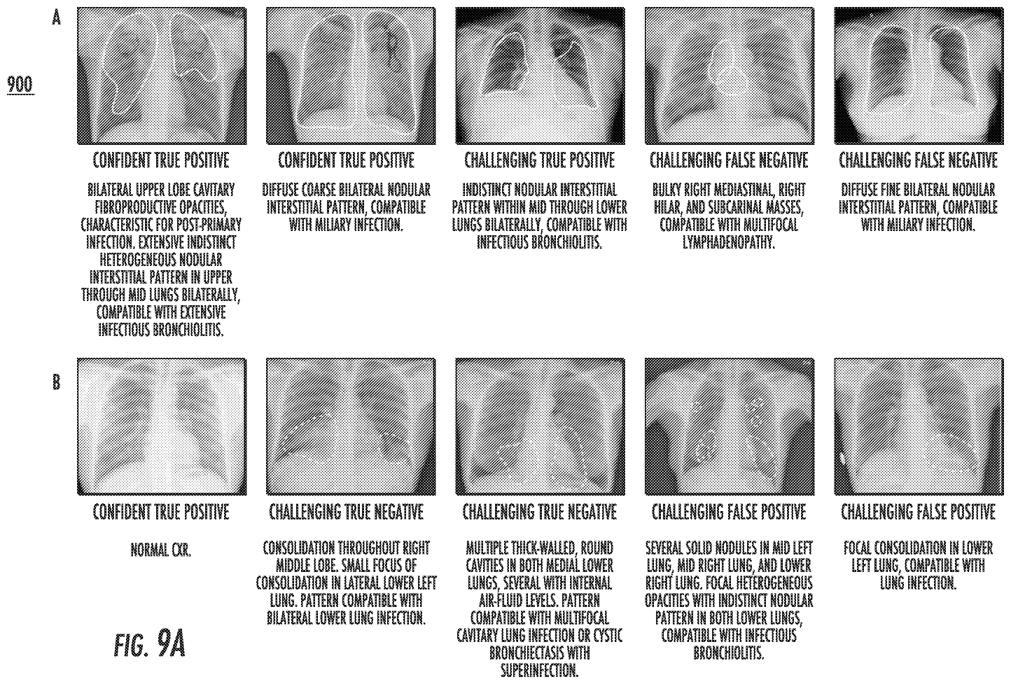

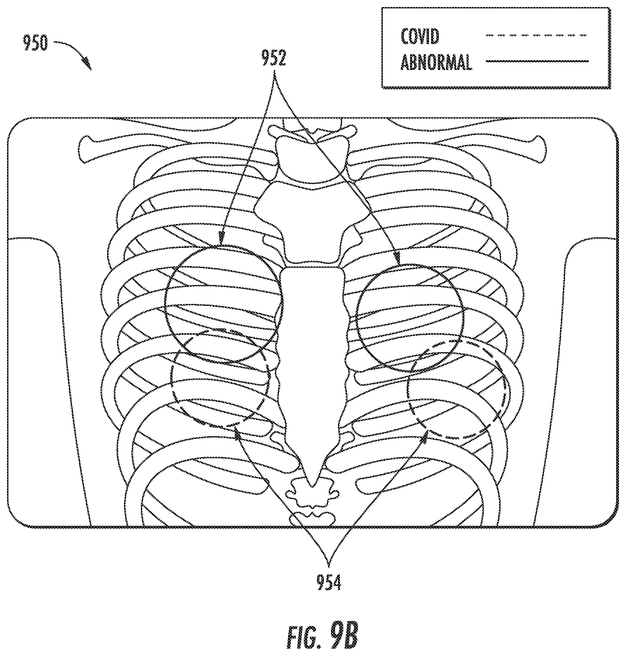

FIGS. 9A and 9B depict example illustrations of attributions overlayed on a chest x-ray of a patient. The attributions can be regions of the chest x-ray that can be used to determine the overall diagnosis or prediction. In some implementations, the attributions overlayed on a chest x-ray of a patient can aid in the prediction of a likelihood of tuberculosis being present. Alternatively and/or additionally, the attributions overlayed on a chest x-ray of a patient can be provided as an output to the patient or a medical professional. The attributions overlayed on a chest x-ray of a patient, or the augmented chest x-ray, can be provided with the tuberculosis diagnosis or as the tuberculosis diagnosis (e.g., FIG. 9B depicts an annotated augmented chest x-ray which includes the tuberculosis diagnosis along with indicators to indicate areas of interest in the patient's lungs.).

FIG. 9A depicts illustrations of various chest x-rays with varying diagnosis with varying levels of confidence 900. The larger shapes in the two top left illustrations illustrate areas of interest. The smaller shapes inside the larger shapes illustrate salient regions. In the depicted illustrations, the salient regions include information that can lead to a positive tuberculosis diagnosis. The other illustrations in FIG. 9A depict other augmented chest x-rays with their diagnosis, level of difficulty, and regions of interest. As depicted in the bottom left illustration, the machine-learned pathology model may determine that no regions of interest exist in the lungs; therefore, the output can include a negative tuberculosis diagnosis with zero annotations on the chest x-ray.

FIG. 9B depicts attributions overlayed on a chest x-ray of a patient, or an augmented chest x-ray 950. In this implementation, the machine-learned pathology model can be trained to also denote and diagnose COVID symptoms. In the depicted augmented chest x-ray 950, the machine-learned pathology model has determined four attributions are present. Two attributions are regions of interest for COVID 954, while two of the attributions are regions of interest for general abnormalities 952. The machine-learned pathology model can provide the augmented chest x-ray and/or a diagnosis to a patient or medical professional.

Example Methods

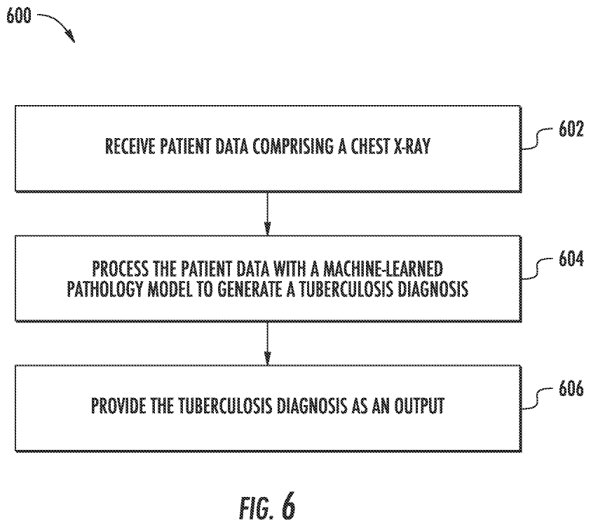

FIG. 6 depicts a flow chart diagram of an example method to perform according to example embodiments of the present disclosure. Although FIG. 6 depicts steps performed in a particular order for purposes of illustration and discussion, the methods of the present disclosure are not limited to the particularly illustrated order or arrangement. The various steps of the method 600 can be omitted, rearranged, combined, and/or adapted in various ways without deviating from the scope of the present disclosure.

At 602, a computing system can receive patient data comprising a chest x-ray. The patient data can be radiograph data. Alternatively, in some implementations, the patient data can be descriptive of medical device imaging of at least a portion of the patient's body.

At 604, the computing system can process the patient data with a machine-learned pathology model to generate risk data predicting a risk that the patient has tuberculosis, such as data constituting a (e.g., preliminary) tuberculosis diagnosis. The pathology model can include one or more machine-learned models, which can include a segmentation model for identifying and segmenting the lungs. In some implementations, the pathology model can include a detection model to detect features in the medical imaging. The output of the segmentation model and the detection model can be processed by a classification model to generate the risk data. The risk data may be descriptive of a tuberculosis positive diagnosis, a tuberculosis negative diagnosis with an abnormality, or a negative tuberculosis diagnosis with normal lungs.

At 606, the computing system can provide the risk data (e.g., the tuberculosis diagnosis) as an output. The risk data (e.g., diagnosis) can be provided directly to the patient or may be provided to a medical professional for review or referral. In some implementations, the risk data (e.g., the diagnosis) can be provided to provide a follow-up action for the user. For example, the pathology model may be used for preliminary screening; therefore, if the diagnosis is tuberculosis positive, the patient may be referred for further testing.

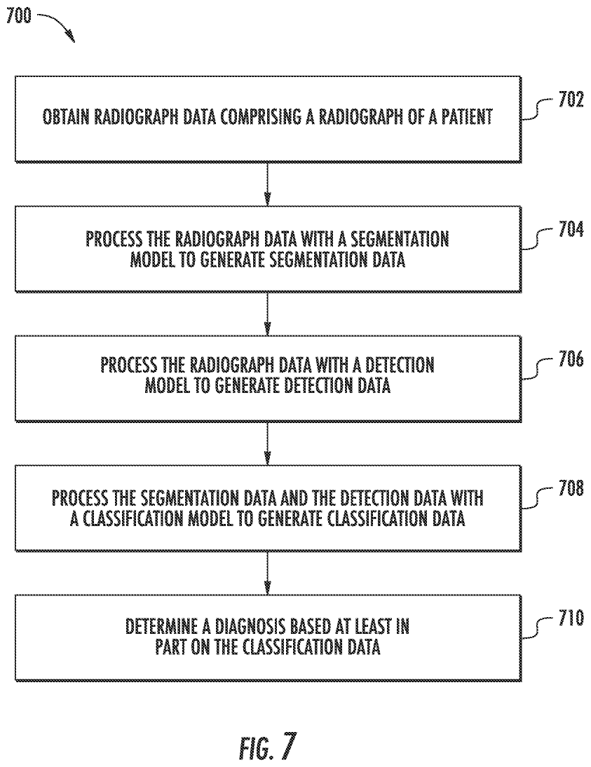

FIG. 7 depicts a flow chart diagram of an example method to perform according to example embodiments of the present disclosure. Although FIG. 7 depicts steps performed in a particular order for purposes of illustration and discussion, the methods of the present disclosure are not limited to the particularly illustrated order or arrangement. The various steps of the method 700 can be omitted, rearranged, combined, and/or adapted in various ways without deviating from the scope of the present disclosure.

At 702, a computing system can obtain radiograph data comprising a radiograph of a patient. The radiograph data can be obtained directly from the medical imaging machine or may be inputted by a user.

At 704, the computing system can process the radiograph data with a segmentation model to generate segmentation data. The segmentation data can be descriptive of a segment of the radiograph that includes the patient's lungs (e.g., not descriptive of parts of the radiograph outside the segment). The segmentation model can include a Mask RCNN.

At 706, the computing system can process the radiograph data with a detection model to generate detection data. The detection data can be descriptive of one or more located features in the chest radiograph. The detection model can include a ResNet architecture and may output bounding boxes and/or attention masks.

At 708, the computing system can process the segmentation data and detection data with a classification model to generate classification data. The classification model can include an EfficientNet block to process the segmentation model and may include an attention pooling block to pool the segmentation data and the detection data. The classification model can include a classification head to classify the pooled data based at least in part on a determined likelihood of a condition or state.

At 710, the computing system can determine a risk data predicting a risk that the patient has medical condition (e.g., a preliminary diagnosis of that condition) based at least in part on the classification data. In some implementations, the classification data can include determined probability of one or more possible conditions or diagnosis. The risk data (e.g., data descriptive of a diagnosis) can be determined based on the classification with the highest probability and/or based on a determined threshold.

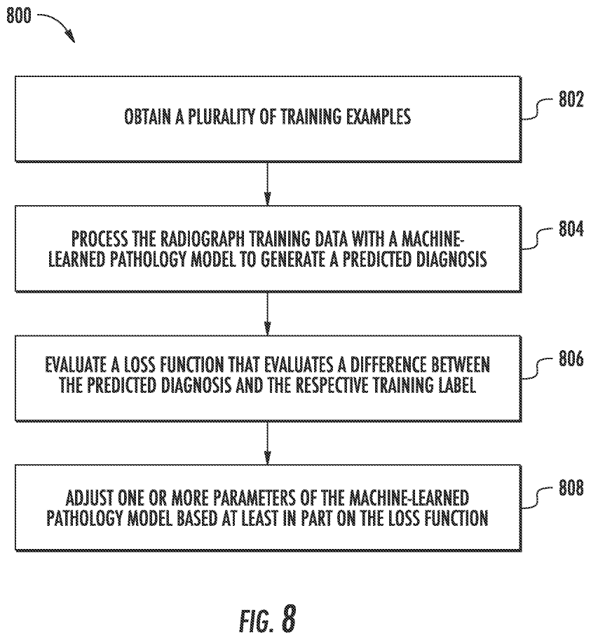

FIG. 8 depicts a flow chart diagram of an example method to perform according to example embodiments of the present disclosure. Although FIG. 8 depicts steps performed in a particular order for purposes of illustration and discussion, the methods of the present disclosure are not limited to the particularly illustrated order or arrangement. The various steps of the method 800 can be omitted, rearranged, combined, and/or adapted in various ways without deviating from the scope of the present disclosure.

At 802, a computing system can obtain a plurality of training examples. The training examples can include radiograph training data and a respective training label for each respective radiograph training data. The training examples can include radiographs pre-labeled by radiologist professionals. In some implementations, the training examples can include training examples from patients from a plurality of countries. In some implementations, the training examples can include training examples descriptive of patients with various medical conditions.

At 804, the computing system can process the radiograph training data with a pathology model to generate risk data, such as a predicted diagnosis. The pathology model can include one or more machine-learned models. The pathology model can include a segmentation model to segment a portion of the radiograph, a detection model to detect features in the radiograph, and a classification model to process the segmentation data and the detection data to generate a risk data (e.g., a predicted diagnosis) based on a determined classification.

At 806, the computing system can evaluate a loss function that evaluates a difference between the risk data (e.g., risk data descriptive of a predictive diagnosis) and the respective training label.

At 808, the computing system can adjust one or more parameters of the pathology model based at least in part on the loss function. The adjustment can be based on differences between the risk data (e.g., risk data descriptive of a predictive diagnosis) and the respective training label. The one or more parameters being adjusted can be one or more parameters of at least one of the segmentation model, the detection model, or the classification model.

Example Implementations and Experiments

The system and method can include one or more machine-learned models trained using chest radiographs (CXRs) from 9 countries spanning Africa, Asia, and Europe. To improve generalization, the system and method can incorporate large-scale CXR pretraining, attention pooling, and semi-supervised learning via “noisy student.” The system can be a deep learning system and can be evaluated on a combined test set spanning sites in China, India, US, and Zambia, all with confirmation via microbiology or nucleic acid amplification testing (NAAT). The India test set can be completely independent of those used in training. An independent test set from a mining population in South Africa can also be used to further evaluate the model. Given the World Health Organization (WHO) targets of 90% sensitivity and 70% specificity, the deep learning system's operating point can be prespecified to favor sensitivity over specificity.

The trained machine-learned models can be tested across 4 countries. In one experimentation, the deep learning system's receiver operating characteristic (ROC) curve was above all 9 India-based radiologists (where TB is endemic), with an area under the curve (AUC) of 0.90 (95% CI 0.87-0.92). At the prespecified operating point, the deep learning system's sensitivity (88%) was higher than the India-based radiologists (mean sensitivity: 75%, range 69-87%, p<0.001 for superiority), and the deep learning system's specificity (79%) was non-inferior to the radiologists (mean specificity: 84%, range 78-88%, p=0.004). Similar trends were observed within HIV positive and sputum smear positive sub-groups and in the South Africa test set. The experimentation results additionally conveyed that 5 US-based radiologists (where tuberculosis is not endemic) who also reviewed the cases were more sensitive but less specific than the India-based radiologists. The deep learning system was similarly non-inferior to this second cohort of radiologists at the same prespecified operating point. Depending on the setting and prevalence, use of the deep learning system as a prioritization tool for NAAT can reduce the cost per positive TB case detected by 40-80% compared to the use of NAAT alone.

In some implementations, the pathology model can include a deep learning system developed to detect active pulmonary tuberculosis on CXRs, which can be generalized to patient populations from 5 different regions of the world and can merit prospective evaluation to assist cost-effective screening efforts in settings with scarce access to radiologists. Operating point flexibility may permit customization of the deep learning system to account for site-specific factors such as tuberculosis prevalence, demographics, clinical resources, and customary practice patterns.

Therefore, the system and method can validate the deep learning system using an aggregate of datasets from China, India, US, and Zambia that together reflect different regions, race/ethnicities, and local disease prevalence. The deep learning system may be evaluated under two conditions: (1) having a single prespecified operating point across all datasets, and (2) when customized to radiologists' performance in each locale. As diagnostic performance may be influenced by disease prevalence, the deep learning system can be compared with two different cohorts of radiologists: one based in a tuberculosis-endemic region (India) and one based in a tuberculosis non-endemic region (United States). An analysis of HIV positive and sputum smear positive subgroups can also be performed. The system and method can be used for cost savings as a triaging solution for nucleic acid amplification testing (NAAT) in screening settings.

In some implementations, the system and method can leverage de-identified C×R images from multiple datasets spanning 9 countries for training and 4 countries for validating the DLS, for a total of 10 countries. The deep learning system can be trained using images from Europe (Azerbaijan, Belarus, Georgia, Moldova, Romania), India, and South Africa, and tuned using images from China, India, and Zambia. Additionally, the system and method can use a plurality of images from a plurality of patients for pretraining purposes, of which a set of the images overlapped with the train or tune sets (though the tuberculosis labels were not used). The deep learning system can then be validated using 1,262 images (1 image per patient) from China, India, US, and Zambia. In some implementations, testing the deep learning system can utilize de-identified images and metadata reviewed by a third party.

For all test and tune datasets, the tuberculosis status can be confirmed via microbiology (sputum culture or sputum smear) or nucleic acid amplification testing (GeneXpert MTB/RIF, Cepheid, Sunnyvale, Calif.). On the training datasets, the reference standard can be varied due to site-specific practice differences and data availability, including microbiology, radiologist interpretation, clinical diagnoses (based on medical history and imaging), and nucleic acid amplification testing.

The pathology model with the deep learning system can be developed to detect evidence of active pulmonary tuberculosis on CXRs. The pathology model can include three models: a segmentation model (e.g., a lung cropping model) for identifying pixels spanning the lungs, a detection model for identifying regions containing possible imaging features of active tuberculosis (nodules, airspace opacities with cavitation, airspace opacities without cavitation, pleural effusion, granulomas, and fibroproductive lung opacities), and a classification model that takes the output from the segmentation model and the detection model to predict the likelihood of the CXR being tuberculosis positive.

For the segmentation model (e.g., the lung cropping model), the system or method can use Mask RCNN with a ResNet-50-FPN backbone architecture. Based on the segmentation results, the system or method can crop each CXR using a tight bounding box enclosing the lungs as the input for the classification model. For the detection model, the system or method can use a Single Shot MultiBox Detector (SSD) to create bounding boxes around potential TB-relevant imaging features. Based on the predicted bounding boxes, a probabilistic attention mask can be calculated as the final pooling layer in the classification model. For the classification model, the system or method can include an EfficientNet-B7 pre-trained on classifying CXRs as normal or abnormal with an attention pooling layer and a fully-connected layer. The attention pooling layer can utilize the probabilistic attention mask generated from the detection model to perform a weighted average of the feature maps before feeding to the final fully connected layer. The classification model can classify CXRs into 1 of 3 classes: tuberculosis-positive, tuberculosis-negative but abnormal, and normal. The system or method can take the prediction score for tuberculosis-positive class as the output prediction for all tuberculosis-related analysis.

In some implementations, training the individual components of the deep learning system described above can be a multi-step process. First, the system can train the segmentation model using lung segmentation masks from the US dataset. To train the detection model, radiologists can be annotated 9,871 bounding boxes around tuberculosis-indicative abnormalities (nodules, airspace opacities with cavitation, airspace opacities without cavitation, pleural effusion, granulomas, lymphadenopathy, and fibroproductive lung opacities). Both the detection and classification models can be trained using the Europe dataset and the two India train datasets. Due to the limited amount of labeled data, the system can use the noisy-student semi-supervised learning approach to leverage a much larger set of unlabeled data. Specifically, the system can obtain “noisy” tuberculosis labels by running inference using the initial version of the deep learning system on the South Africa (Driefontein) dataset with more than 150,000 unlabeled CXRs. These datasets with generated labels can be combined with the original dataset to train 6 classification models, which can then be ensembled by taking the mean of the scores.

For the detection model, the system and method can use a dropout keep probability of 0.99, and augmentation included random cropping, rotation, flipping, jitter on the bounding boxes, multi-scale anchors, and a box matcher with intersection-over-union. For the classification model, the system or method can apply dropout, with a dropout keep probability of 0.5. Furthermore, the system or method can apply data augmentation such as horizontal flipping, random shears, and random deformations. All hyperparameters can be selected based on empirical performance on the tune sets. Training can be done using TensorFlow on third-generation tensor processing units with a 4×4 topology. All images can be scaled to 1024×1024 pixels, and image pixel values can be normalized on a per-image basis to be between 0 and 1.

For model selection (checkpoint selection and other hyperparameter optimization), the system or method can include models to maximize the area under the receiver operating characteristic curve (area under ROC curve, or AUC) corresponding to the range of radiologists' sensitivities in the tune sets. The approach can be used to help explicitly select models that can be a performant across the range of radiologist sensitivities, instead of potentially optimizing for ranges that can be beyond the scope of customary clinical practice.

In order to gauge the performance of the deep learning system across datasets containing cases of different levels of difficulty, all test set cases can be reviewed by a team of radiologists, whose performance not only served as a baseline for comparison, but also as an indirect indicator of difficulty level. As the performance characteristics of radiologists accustomed to practice in endemic vs. non-endemic settings can vary, the team can include two cohorts of radiologists (10 India-based consultant radiologists and 5 US-based board-certified radiologists). The India-based radiologists had an average of 6 years of experience (range 3-9), while the US-based radiologists had an average of 10.8 years of experience (range 3-22). The radiologists were provided with both the image and additional clinical information about the patient when available (age, sex, symptoms, and HIV status), whereas the deep learning system can be blinded to the information. For each image, the radiologists labeled the radiograph data or images for the presence/absence of tuberculosis, other pulmonary findings, and optionally whether the data may have any minor technical issues visible on the image. The tuning sets can be labeled similarly, by the India-based radiologists and the US-based radiologists.

A trained pathology model can be compared against the performance of the India-based radiologists on the pooled combination of 4 test datasets. To support comparisons with the binary judgments of experts, the system or method can include a continuous score using an operating point of 0.45, chosen based on an analysis of the tune datasets (conducted prior to evaluating the deep learning system on any of the test sets).

In particular, the sensitivity and specificity of the pathology model and the pathologists can be compared, both with a 10% absolute margin. To account for correlations within case and within radiologist, the testing can involve the use of the Obuchowski-Rockette-Hillis procedure configured for binary data and adapted to compare readers with the standalone algorithm in a noninferiority setting. A p-value below 0.0125 can be considered significant for the primary analyses. Subsequent superiority testing can be prespecified if non-inferiority was met, which does not require multiple testing correction.

Prespecified secondary analyses can include per-dataset subgroup analysis for ROC; sensitivity and specificity at the prespecified operating point; operating points corresponding to the WHO thresholds; matched sensitivity/specificity analysis on a per-dataset and per-radiologist level; comparisons of the India-based and US-based radiologists, and comparison of the DLS to the US-based radiologists. Additional secondary analysis can be subgroup analyses across HIV status, images flagged by the reviewing radiologists to have minor technical issues, demographic information, and symptoms. Exploratory subgroup analysis can be based on sputum smears conducted. Unless otherwise specified, 95% confidence intervals (CIs) can be calculated using the bootstrap method with 1000 samples.

In some implementations, to understand the performance of the “abnormality” detector in the deep learning system (DLS), the India test dataset can be labeled for any actionable abnormal CXR findings. Each case may be reviewed by three US-based radiologists. Because follow up testing such as a repeat CXR or computed tomography may not be available, “ground truth” can be based on how many radiologists indicated the presence of an abnormal finding: at least 1 of 3, at least 2 of 3, and all 3 of 3.

Additionally and/or alternatively, the systems and methods can improve cost efficiency. To test the cost efficiency of one implementation, an experimentation set was used to simulate the potential cost savings of using the pathology model as a tuberculosis screening intervention. Recent studies have estimated the overall cost for subsidized GeneXpert to be about US$13.06 per test, including equipment, resources, maintenance, and consumables. The cost to acquire a single digital CXR was estimated to be US$1.49, including equipment and running costs, but not radiology interpretation. In the simulation, the pathology model can be used for initial tuberculosis screening, and patients who meet the threshold (based on our prespecified operating point) can proceed with GeneXpert testing. The cost of CXR screening for all patients and the cost of GeneXpert testing for these DLS-positive patients was computed and divided the total cost by the number of true positive tuberculosis cases caught to derive the cost per positive tuberculosis case. The experimentation results were then analyzed to understand the effect of prevalence on the cost, which makes the simplifying assumption that there are no changes in case severity or other factors that may affect deep learning system performance.

For one implementation of the deep learning system, the performance was evaluated on 4 datasets incorporating a diverse population representing multiple races and ethnicities, drawn from 4 countries: China, India, US, and Zambia. Among a total of 1,262 images from 1,262 patients, there were 217 tuberculosis cases based on positive culture or GeneXpert. The deep learning system development (training and tuning) and operating point selection was conducted on the tune datasets, independently of the test datasets. Patient sources for these 4 datasets included tuberculosis referral centers, outpatient clinics, and active case finding. The India test dataset was from a site independent of those used in development.

In the combined test dataset across 4 countries, the deep learning system achieved an AUC of 0.90. To contextualize the model's performance and better understand the case spectrum, the obtained radiologist interpretations for the same cases from two cohorts of radiologists included: radiologists based in India, a country where tuberculosis is endemic, and radiologists based in the US. One India-based radiologist was found to have a rate of flagging positives (and consequently sensitivity) substantially below the others and so was excluded from subsequent analyses to avoid under-representing radiologist performance. The deep learning system's ROC curve was above the performance points of all 9 remaining India-based radiologists.

One set of analyses involved comparisons of the deep learning system at a prespecified operating point (0.45) with India-based radiologists. The deep learning system's sensitivity (88%, 95% CI 83-94%) was higher than (superiority test was conducted if non-inferiority passed) the India-based radiologists (median sensitivity: 74%; IQR: 72-76%), p<0.001. At the same operating point, the deep learning system's specificity (79%, 95% CI 75-82%) was similarly non-inferior to the India-based radiologists (median specificity: 86%; IQR: 81-87%), p=0.003.

While both India-based and US-based radiologists had sensitivities and specificities that tracked closely and slightly below the ROC curve of the example pathology model, the conservativeness with which the two groups of radiologists called cases as positive for tuberculosis appeared to differ. India-based radiologists appeared to be more specific but less sensitive than US-based radiologists, who had a median sensitivity of 84% (IQR 76-86%) and a median specificity of 71% (IQR 67-81%). The deep learning system's sensitivity and specificity remained comparable to the US-based radiologists (p-value for non-inferiority: 0.022 for sensitivity; 0.018 for specificity).

Subgroup analysis can then be conducted on a per-dataset level. The China and US datasets can be similarly-constructed case-control datasets, with normal CXRs selected to match the tuberculosis positive CXRs. On these two datasets, while the India-based radiologists achieved high specificity (96-99%), their sensitivity was lower (53-65%) compared to in the combined dataset. At the prespecified operating point of the example pathology model, both the deep learning system's sensitivity and specificity were non-inferior to the radiologists in both datasets (p<0.001 for all 4 comparisons). In the India dataset, which consisted of tuberculosis presumptive patients can be identified in a tertiary hospital, the deep learning system can be similarly non-inferior in both sensitivity and specificity (p<0.001 for both). In the Zambia dataset, which was taken from a trial, nucleic acid amplification testing can be associated with cases where the CAD4 TB system had flagged an abnormal CXR, resulting in substantial enrichment for CXR-abnormal TB-negatives. In the dataset, at the prespecified operating point, the deep learning system can be non-inferior for sensitivity (p<0.001) but not for specificity (p=0.504), though 8 of 9 India-based radiologists were below the ROC curve.

In addition to the 4 datasets above, one implementation of the deep learning system was evaluated on another independent dataset from a mining population in South Africa. The ROC curve of the model was above all but 1 radiologist. At the same prespecified operating point as the other datasets, the deep learning system was non-inferior both in terms of sensitivity and specificity to both India-based and US-based radiologists (p<0.05 for all). At a higher (lower sensitivity) operating point selected based the South Africa tune datasets, the deep learning system was again non-inferior in both sensitivity and specificity compared to the India-based radiologists but had higher specificity (p=0.012) at the cost of not being non-inferior in sensitivity (p=0.571).

To better understand inter-dataset differences, histograms of one implementation of the deep learning system's prediction scores were plotted separately for tuberculosis positive and tuberculosis negative cases for each dataset. The distribution of deep learning system scores for both tuberculosis positive and tuberculosis negative cases remained similar across the China, India, and US datasets. However, the dataset had a higher proportion of tuberculosis-negative cases with high deep learning system scores in the Zambia dataset. The trend appears to have been a consequence of first-round computer aided detection screening of the Zambia dataset which censored a plurality of normal-appearing CXRs, resulting in a more challenging dataset with a relative paucity of normal CXRs.

To facilitate comparisons despite the wide range in radiologists' sensitivities and specificities, both across datasets and readers, a matched analysis can be conducted by shifting the deep learning system's operating point on a per-dataset level to (1) compare sensitivities at mean radiologist specificity, and (2) compare specificities at mean radiologist sensitivity. The analyses can be done separately for the India-based radiologists and US-based radiologists, for a total of 16 analyses (4 datasets*2 comparator radiologist group*matching sensitivity/specificity). The deep learning system can have non-inferior performance in 15 out of these 16 analyses (p<0.05 for 15 and p=0.068 for the remaining).

In some implementations, the deep learning system's operating point can be adjusted to match each individual radiologist's sensitivity and specificity, focusing on the two larger datasets (India, Zambia) to improve statistical power. With 14 radiologists, 2 datasets, and matching sensitivity/specificity, this amounted to 56 analyses. In 50 of these analyses, the deep learning system can be non-inferior (p<0.05), with 4 of the 6 non-passing tests in the enriched Zambia dataset and in comparison with US-based radiologists.

The World Health Organization (WHO) “target product profile” for a tuberculosis screening test recommends a sensitivity ≥90% and a specificity ≥70%. To further understand the performance of one implementation of the deep learning system, an experiment can be conducted, such as matched performance analysis, similarly to the radiologist-matched analysis above. At 90% sensitivity, the deep learning system can have a specificity of 77% on the combined dataset; and at 70% specificity the deep learning system can have a sensitivity of 93%, both of which met the recommendations. This remained true in the China, India, and US datasets, but not in the enriched Zambia dataset.

Subgroups based on HIV status can be considered where available. The deep learning system can adjust for the challenge that is the HIV-positive subgroup being more challenging than the HIV-negative subgroup (DLS AUC: 0.81 vs 0.92), and a similar lowering of sensitivity and specificity were observed for the radiologists. The example implementation of the deep learning system remained comparable to the radiologists in both subgroups, notably despite the deep learning system not having access to the HIV status as the radiologists did.

Sputum smear microscopy can be fast, inexpensive, and specific for Mycobacterium tuberculosis. Despite the low sensitivity, sputum smear microscopy can be used for rapid diagnosis in resource limited settings. The performance of the example model can be evaluated on this subset using a sample dataset and can evaluate the deep learning system's sensitivity for tuberculosis-positive cases with different sputum smear results. Although this subset was small with only 12 smear positive and 14 smear negative patients, at the example prespecified operating point, the example model had 100% sensitivity for smear-positive tuberculosis-positive patients and 71% sensitivity in smear-negative tuberculosis-positive patients.

In some implementations, the “abnormality” detector in the deep learning system can be evaluated using indications of abnormal findings on CXR by three US-based radiologists as the “ground truth” (Methods). The deep learning system can then be evaluated using this ground truth in two ways. First, the system can define a positive case as either being TB-positive, or having another abnormal finding, and summed the DLS's TB prediction with its “non-TB abnormality” prediction per case to plot the ROC. Depending on how many radiologists may have indicated the presence of the abnormality, the DLS's AUC ranged from 0.80 to 0.96. Second, the system can remove all TB-positive cases and define the positive cases as having any abnormal finding, and plotted the ROC for the DLS's “non-TB abnormality” prediction alone. The AUC of the DLS can range from 0.71 to 0.85, though with wider confidence intervals.

In an analysis of potential cost savings, a workflow can be simulated where patients only proceed to GeneXpert testing if they are flagged as positive by the deep learning system. The workflow can have a reduced overall sensitivity (with WHO targets being 90%), but substantially reduces cost via a lower number of rapid molecular tests being conducted and thus improves cost effectiveness as measured by cost per positive tuberculosis case detected. Based on the performance on the India dataset (94.11% sensitivity and 95.11% specificity), as prevalence decreases from 10% to 1%, the cost per positive tuberculosis case detected can increase substantially, and the cost savings compared to using GeneXpert alone can increase from 73% to 82%. The corresponding cost savings can increase with prevalence decreases when simulating the WHO target (90% sensitivity and 70% specificity) was 47% to 53%, and when simulating a lower-specificity device (90% sensitivity and 65% specificity) was 42% to 48%.

In order to achieve the long term public health vision of global elimination of tuberculosis, there is a pressing need to scale up identification and treatment in resource-constrained settings. The recently-released 2021 WHO consolidated guidelines stated that CAD technologies had the potential to “increase equity in the reach of TB screening interventions and in access to tuberculosis care.” They also emphasized the importance of using a high-performing CAD that was tested on CXRs drawn from a representative population for the corresponding use case. Some implementations of the deep learning system can be, and have been, developed using data from 9 countries and validated in 4 countries, together covering many of the high-tuberculosis-burden countries and a wide range of race/ethnicities and clinical settings. In this combined international test dataset, the example deep learning system's pre-specified operating point demonstrated higher sensitivity and non-inferior specificity relative to a large cohort of India-based radiologists. The development of a deep learning system with robust performance across a broad spectrum of patient settings can have the potential to equip public health organizations and healthcare providers with a powerful tool to reduce inequities in efforts to screen and triage tuberculosis throughout the world.

When considering each dataset individually, the example deep learning system's performance was excellent in two commonly-used case-control datasets from China and US and generalized well to an external validation set in India. Moreover, the example deep learning system's performance was maintained in the enriched Zambia dataset, which was filtered by another CAD device. Since many images that were considered radiologically clear were excluded from this dataset, the difficulty of triaging the remaining cases was likely increased. The example deep learning system also performed well when radiologists indicated minor technical issues with the image, indicating robustness to real-world issues.

In addition to performing well in different countries with a wide range of race/ethnicities, the example model was also comparable to radiologists in important subgroups. First, HIV infection increases the risk of active tuberculosis disease up to 40 fold compared to background rates. Patients with HIV-associated pulmonary tuberculosis can often have an atypical presentation on CXR, making them more difficult to screen. Thus, the fact that the example deep learning system's detection performance remained comparable with radiologists in HIV-positive patients is reassuring. Second, sputum smear can have a fast turnaround and a low cost, leading to its importance in resource-limited settings despite having limited sensitivity. Though the subgroups were small, the example deep learning system was able to identify all sputum-positive cases and remained accurate on sputum-negative cases. Finally, the deep learning system can be able to accurately detect other non-TB abnormalities that were identified by radiologists. Such a capability can resolve one of the drawbacks that traditional CAD systems were noted to have by the WHO: that unlike human readers, the CAD systems may not simultaneously screen for pulmonary or thoracic conditions.

Although diagnostic systems such as GeneXpert can have high positive predictive value, many populations may be unable to derive the broadest possible benefit from such tests because of their higher relative per-unit cost. However, if coupled to an inexpensive but relatively sensitive first-line filter like CXR (i.e., only cases screening positive on CXR are tested using mWRD (i.e., molecular WHO-recommended rapid diagnostic test)), the benefits of mWRDs may effectively benefit a larger population due to more targeted use. Two-stage screening strategies of this type can traditionally be intractable in many locales because settings with constrained access to mWRDs often also lack providers trained to reliably interpret CXRs for TB-related abnormalities. In these settings, in accordance with current WHO guidelines, a robust performing CAD can increase the viability of this strategy by serving as an effective alternative to human readers. The cost analysis of this two-stage screening workflow using the DLS can suggest that it has the potential to provide 40-80% cost savings at 1-10% prevalence. The cost savings can increase further as prevalence falls, which can be an important financial consideration in disease eradication.

The analysis with a large cohort of radiologists revealed several important subtleties. First, radiologists irrespective of practice location demonstrated a wide range of sensitivities and specificities. For example, even among the 9 India-based radiologists, sensitivities spanned a 18% range (69-87%). The variation means that direct, single-operating-point comparisons with any individual radiologist can be difficult to interpret without matching the deep learning system operating point to either the sensitivity or specificity of that reader. Second, radiologists practicing in India were generally more specific and less sensitive than those practicing in the US. The discrepancy may partially be due to practice patterns: in India where TB is endemic, radiologists' calls need to be highly specific to avoid testing an overwhelming number of patients. By contrast, in the US where TB is relatively rare and the goal is to avoid outbreaks, radiologists are incentivized to make calls that are highly sensitive at the expense of specificity. Therefore, the practice locations of implementation can be important for adjusting the sensitivity and specificity of the implemented model.

Remarkably, despite variability in individual radiologists' sensitivity and specificity, the performance tracked closely with the example deep learning system's ROC curve, with the clearest evidence of this trend seen in an enriched Zambia dataset which exhibits a marked rightward shift toward lower specificity. The trend can suggest that the inherent advantageous ability of the deep learning system to provide continuous “scores” as output for thresholding can likely help individual sites customize the triggering rate to their local practice patterns and resource needs, while trusting that the customized operating point has a similar effect to calibrating the “conservativeness” of a radiologist. The ability to calibrate the operating point may be critical over time even for the same population, as prevalence and disease severity changes over time. Statistical methods to tune operating points for each dataset and to detect when operating points may be updated over time to be useful in this regard and can be an important direction for real-world use cases.

Example Datasets

The training dataset and/or the testing datasets can include data collected from a variety of resources and regions.

| Dataset/ | | | |
|---|---|---|---|
| Location | Description | Tests | Label Criteria |
| South Africa | Data collected from periodic employee | GeneXpert | Positive: |
| (various | screening provided by a Gold Mining | Culture | Positive TB test (GeneXpert, Culture, |
| locations) | Company. Patients suspected of having TB | Smear | Smear) within +/−45 days of CXR. |
| | based on clinical assessment and | | No negative GeneXpert or sputum |
| | radiographic evidence can receive | | culture within +/−150 days of CXR. |
| | GeneXpert and/or culture testing. | | Negative: |
| | | | Negative GeneXpert or sputum culture |
| | | | test within +/−150 days of the CXR. |
| | | | Never registered in the company TB |
| | | | register. |
| Lusaka, | This dataset can include patients who were | GeneXpert | Positive: |
| Zambia | enrolled in a TB outreach study involving | Culture | Positive TB test (GeneXpert, Culture, |
| | case finding at healthcare facilities and in | Smear | Smear) |
| | the community. When CXR was available, | | Negative: |
| | all patients may have been screened with | | Negative TB test (GeneXpert, Culture) |
| | CAD4TBv5 followed by GeneXpert for | | Excluded: |
| | patients with abnormal CXRs and at the | | People screened by CAD4TBv5 who did |
| | discretion of the clinician. When a CXR was | | not get a GeneXpert or Culture. |
| | not available, symptomatic patients may be | | Patient with TB trace results from |
| | asked to provide sputum for GeneXpert. | | GeneXpert test. |
| Shenzhen, | This public dataset can include a curated | Not | Positive: |
| China | subset of TB case-control patients who | described | Positive TB test (e.g., molecular or |
| | presented to a Shenzhen Hospital for routine | | culture). |
| | care. | | Negative: |
| | | | Radiologically clear or negative culture. |
| India | Private tertiary Indian hospital: | NAAT | Positive: |
| | This dataset can include patients who | | Positive rapid molecular testing. |
| | presented to the tertiary hospital with | | Negative: |
| | symptoms suggestive of TB. | | Patients without clinical suspicion of TB |
| | Primary health clinics: | | or having negative rapid molecular |
| | This dataset can include patients who | | testing. |
| | presented to community clinics with | | |
| | symptoms suggestive of TB. | | |
| Montgomery, | This public dataset can include a curated | Culture | Positive: |
| Maryland, US | subset TB case-control patients who were | | Positive TB test (e.g., molecular or |
| | part of Montgomery County's Tuberculosis | | culture). |
| | screening program. | | Negative: |
| | | | Confirmation not documented. |
| Chennai, India | This dataset can include patients referred to | NAAT | Positive: |
| | a private Indian hospital for TB testing | | Positive rapid molecular testing. |
| | based on symptoms or exposure. | | Negative: |
| | | | Negative rapid molecular testing. |

Additional Disclosure

The technology discussed herein makes reference to servers, databases, software applications, and other computer-based systems, as well as actions taken and information sent to and from such systems. The inherent flexibility of computer-based systems allows for a great variety of possible configurations, combinations, and divisions of tasks and functionality between and among components. For instance, processes discussed herein can be implemented using a single device or component or multiple devices or components working in combination. Databases and applications can be implemented on a single system or distributed across multiple systems. Distributed components can operate sequentially or in parallel.

While the present subject matter has been described in detail with respect to various specific example embodiments thereof, each example is provided by way of explanation, not limitation of the disclosure. Those skilled in the art, upon attaining an understanding of the foregoing, can readily produce alterations to, variations of, and equivalents to such embodiments. Accordingly, the subject disclosure does not preclude inclusion of such modifications, variations and/or additions to the present subject matter as would be readily apparent to one of ordinary skill in the art. For instance, features illustrated or described as part of one embodiment can be used with another embodiment to yield a still further embodiment. Thus, it is intended that the present disclosure cover such alterations, variations, and equivalents.

1. A computer-implemented method, the method comprising:
receiving, by a computing system comprising one or more processors, patient data comprising a chest x-ray of a patient;
processing, by the computing system, the patient data with a machine-learned pathology model to generate risk data predicting a risk that the patient has tuberculosis; wherein the machine-learned pathology model is trained to segment chest x-ray data and generate the risk data based at least in part on a segmented portion of the chest x-ray data; and
providing, by the computing system, the risk data as an output.

2. The computer-implemented method of claim 1, wherein processing, by the computing system, the patient data with the machine-learned pathology model to generate the risk data comprises:
processing, by the computing system, the patient data with a segmentation model to generate pixel data descriptive of identified pixels found corresponding to a patient's lungs;
processing, by the computing system, the patient data with a detection model to generate detection data descriptive of regions of the chest x-ray with detected features; and
generating, by the computing system, the risk data with a classification model based at least in part on the pixel data and the detection data.

3. The computer-implemented method of claim 1, wherein the machine-learned pathology model comprises an attention pooling sub-block.

4. The computer-implemented method of claim 1, wherein the machine-learned pathology model is trained to have at least 90 percent sensitivity and at least 70 percent specificity.

5. The computer-implemented method of claim 1, wherein the machine-learned pathology model comprises a deep learning system architecture trained on a plurality of training examples from a plurality of patients from a plurality of countries.

6. The computer-implemented method of claim 1, further comprising:
determining, by the computing system, a follow-up action based at least in part on the risk data.

7. The computer-implemented method of claim 1, wherein processing, by the computing system, the patient data with the machine-learned pathology model further comprises:
determining, by the computing system, attributions in the patient data; and
overlaying, by the computing system, the attributions on the chest x-ray of the patient to provide visual cues of suspicious areas.

8. The computer-implemented method of claim 1, wherein the risk data is indicative of at least one of positive, negative, or non-tuberculosis abnormality.

9. A computing system, the computing system comprising:
one or more processors;
one or more non-transitory computer readable media that collectively store instructions that, when executed by the one or more processors, cause the computing system to perform operations, the operations comprising:
obtaining radiograph data comprising a radiograph of a patient;
processing the radiograph data with a segmentation model to generate segmentation data descriptive of a segment of the radiograph comprising one or more organs;
processing the radiograph data with a detection model to generate detection data descriptive of one or more located features in the radiograph;
processing the segmentation data and the detection data with a classification model to generate classification data, wherein the classification data is descriptive of a determined classification for the one or more located features; and
determining risk data based at least in part on the classification data.

10. The computing system of claim 9, wherein processing the segmentation data and the detection data with a classification model to generate classification data comprises:
processing the segmentation data and the detection data with an attention pooling sub-block to generate pooled data; and
processing the pooled data with a diagnosis model to generate the risk data, wherein the diagnosis model comprises a convolutional neural network.

11. The computing system of claim 9, wherein the segmentation model comprises a mask region based convolutional neural network.

12. The computing system of claim 9, wherein the detection model comprises a residual neural network architecture.

13. The computing system of claim 9, wherein the classification model comprises a convolutional neural network comprising a fully connected sub-block.

14. The computing system of claim 9, wherein at least one of the segmentation model or classification model comprises an EfficientNet architecture.

15. The computing system of claim 9, wherein the segmentation data comprises a feature map.

16. The computing system of claim 9, wherein the detection data comprises one or more attention masks.

17. One or more non-transitory computer readable media that collectively store instructions that, when executed by one or more processors, cause a computing system to perform operations, the operations comprising:
obtaining a plurality of training examples, wherein each training example comprises radiograph training data and a respective training label, wherein the radiograph training data and the respective training label are descriptive of a patient with a tuberculosis diagnosis;
processing the radiograph training data with a machine-learned pathology model to generate risk data, wherein the machine-learned pathology model is trained to segment radiograph data and generate risk data based at least in part on segmented radiograph data;
evaluating a loss function that evaluates a difference between a predicted diagnosis associated with the risk data and the respective training label; and
adjusting one or more parameters of the machine-learned pathology model based at least in part on the loss function.

18. One or more non-transitory computer readable media of claim 17, wherein the training examples comprise a set of region-specific training examples.

19. One or more non-transitory computer readable media of claim 17, wherein the training examples comprise a set of HIV training examples and a set of non-HIV training examples, wherein the set of HIV training examples comprises one or more tuberculosis positive examples and one or more tuberculosis negative examples.

20. One or more non-transitory computer readable media of claim 17, wherein:
the machine-learned pathology model comprises a segmentation model, a detection model, and a classification model; and
wherein adjusting one or more parameters of the pathology model based at least in part on the loss function comprises adjusting one or more parameters of at least one of the segmentation model, the detection model, or the classification model.
